SEQUENCE LISTING (95 sequences)

SEQ ID NO: 1 - DNA, 324 bp, synthetic construct
  source 1..324: mol_type = other DNA
atgaataaattaatatccgccttatttgcagcttgtgttttggctgcctgcgcatcgccgaactctgccggcaatatggcgatcggcgatgattctgccgccattaaggcgggaagaaaccgaatgtctgccgagttgagccggtctgaattggagcagcagcgccgccaaagaaccaacacggcagaggaaatcgctttagaggagcaaaagcgcagaaacaaacacgaaggcatcagggataccatgcgcaccacttcgggcggcctgggtgtgattaatgaggcaatcggcaccatacgcaacctacgctggggcttttaa

SEQ ID NO: 2 - DNA, 255 bp, synthetic construct
  source 1..255: mol_type = other DNA
atttttgtgaacaatttgttaatccccgctttcgctgccgtgctgctgcttggcggctgcacttatgccgaccaacgctttgtttcctcccaatcccagcaacagcagctttatgaaaacaggctcgctatccagaaagcagagcgggaggagcgggctgagcgcagagccgaacgccgcgaagagatgatggatgaggccgatgccgtcaaccgcgcatataaagggcttaacggagtttaccttatccgctga

SEQ ID NO: 3 - DNA, 411 bp, synthetic construct
  source 1..411: mol_type = other DNA
atgtttaaacgccctgaagagttgattgtggctgtgctggccgccttgtgggtggtgttggcctatctggccgccgattatctcggtgcccccgcgcaaaccaagttgctgatttctgcgctgacgctgatttgtaccgccctgtgcttcatactgtggcagcgcaacctgagccgggcggtttggccgttttttctgggaatgctgaccgcctgttggtggccttatctcgattggatggcggtgcgcgatattctggtgccgggcgcagagtcttcggccattatcatcaacaagccctggtatgcgggctggacttttaaaaccattcttgccgccgcgcccgtggtgctgggctatgcggtaaagtggaaactgcacagcaaacacaagcataaagcggtggattag

SEQ ID NO: 4 - DNA, 603 bp, synthetic construct
  source 1..603: mol_type = other DNA
atggccggttgggatgtatggatttcggttgtttatattgccgtgtttttcaccgctgcttttaaagcgggagagtttcaatggctgtggggaagcgtgctgctctggtttgggttcggccttgccggcgcacgccttctgcctggaatatggggtatcatccacttggctccgctttatcctccccatctctatatcataccggccagcctgtttttctttgcaaaccattggaaaaaagtgcctgaaaagaagatgtggcaggccgaaaatgccaacggttttatcagcttgtttgcagtaagcggtatgctgatgggcatcgtgtctgctgtgttggcgttagtggtgctcagccgctttccggcgggcataaccccttatgttttgcctgtgctgctgcagctttatgctttcgaaccggcgtattggttcggaatgcaggctgtgatcatggtggttttttatctgcaccgcaaagttattgcgggcgaggcggcaaaccgtttcagccgcctccagctgcaactgggttttctgttggccttattgtttcagacggcctgcacagttgtattgctgtcagatatacgttacggttga

SEQ ID NO: 5 - DNA, 489 bp, synthetic construct
  source 1..489: mol_type = other DNA
atgaaaaaatacctgccctttttacgttttgtgctgattgcctgtatatcggcattcttggcgtggaaagggcagccttttgttcatggaaacgagaaagcagttgatttaattatcaacgtatttgcaatcttggccggttttctgattgcgattatgacgctgtttagtgatatgcggtttgatgaagatgcgaattggcggcaaattcaaattcgtgaaggcgtacaagaacaaagatatatcaaacattccatgctcttttacacttatctggcagtattggtttgtgtttttcttgttatattgttggctcataaagaagaatataaaaatggacaaataattttttggctagagcggggatatttattcttagcctgcatgtccattttttactctgtatttttacccggaaacctaattaaaagccggaaagaagagtttcaaaaactcatggataaaaagaaacccaaatatccgaattga

SEQ ID NO: 6 - DNA, 288 bp, synthetic construct
  source 1..288: mol_type = other DNA
atgaataaagaattgctcggcattgtttttctgcccgccggcgtcatcagcatgtgcatggcggcattgtggcagatgtatgtggtgatcaccgaaagctacatgcttaaccgcttcaaagacaagcaattggtgtgggtagtggccgtgctgtttttcagcttcagcactgccgtttactggttttgcccgaatgcgcgcaaaaaaggcatattgtttctgctgctgggtgcgggcggcgcggtgatgtatggcttggccagaatatggctgccgttcggcaaataa

SEQ ID NO: 7 - DNA, 438 bp, synthetic construct
  source 1..438: mol_type = other DNA
ttgaaactcaatcgaaaacaagccgtttgggcgggcatattgctgttggcgtttgccgccgtaaaaatagcggcactgctgtggtggcagagccgccagcccgccgttacctccgttgccgaatctgcctgcaacgtacacgcgggctgcacgctgcccaacggagcgcaagtgaaattcagcagcaaagtggccgccaaagcgccgttcgacatcaccctgcacaacgtgccggcgcaaacggccgaagtgtttgtgagtttttcgatgcgcggtatggacatgggcttcaaccgctacaaactgctgcgccggcctgacggctcgtgggcggcaccgcaaatccgtctgcccgtgtgcgtgcagaaccgccgtgattatctggccgacatccatatcggcggcgaggtgttccaagtgggctttaccgccgagtga

SEQ ID NO: 8 - DNA, 276 bp, synthetic construct
  source 1..276: mol_type = other DNA
atgcaaagcacgcaacgcaaaggtttggtgttggtcagcacattggccgcaatggctgcagcattctcttccggccacacggaggcccaatccgccgaaatgccgctgtcggagttgggctgcgataaattgaatgtattgcgaatcacgcaaatgaacggtaaacaacaagtttttgcacgcgaatgcgccgctgcagaagccgcccaagcatgggaacagtcttacggcgcacttgactccgaaagtttgattgcgagtgtgatatacgaatag

SEQ ID NO: 9 - DNA, 447 bp, synthetic construct
  source 1..447: mol_type = other DNA
atgagcgtttattctgttgcacatatcatacatttattttgcgccatcgtttttgtgggcggcgttttctttgaaacgctggtgttgtcggctatacacagcaaagatgtaccgagggaggcgcgcagaacggtagaaaaagccattatgcggcgggcggtgcgggtgatgccgtgggtggtgggcagtgtgtttctgtcggggttgacaatggcacaccgttatgctgccgcattcgccgcaccattttccgccccgttcaacactcagctgtgtttgaaagcgttgctggcgtttggtgtgttagcgcactttctgattgccataatcaaaatgcggcgcaatacactcacagcagcttggtcaaaatatatccatctggcggtattttgccagatggtgttgattgtgcttctggcgaaaaccatgttttatatagcttggtag

SEQ ID NO: 10 - DNA, 261 bp, synthetic construct
  source 1..261: mol_type = other DNA
atgaaaaccacaacttctactgctttggccgcattggccggtgcgctggcactggcgggttgtcaggccggttctgccccctctgctccgattgcagcctctgggcagcataaagtgggcgagggaaaatgcggcgcaagcggcaaagctgccgaaggtaaatgcggcgcaacatcgcacaatggcaaggctgccgaaggcaaatgcggcgcagcatcgcacagcggtaagtctgccgaaggtaaatgcggcagtaaatga

SEQ ID NO: 11 - DNA, 444 bp, synthetic construct
  source 1..444: mol_type = other DNA
atgcacattacccgcaccgtctatactgccgctcttctctgcctgctgcctgcgcaatttgccgctgcccagatttacacctggaaaggcaaaaacggcagcaccagttattcagatgtgccgcacaacctgaaaatttccaacgcatcaaccatcaacgtgcgcacccgcaccgttaccccgccgcccgccgcccaaaccgcagaaagcgcaggcgaaggctcgctggccgaccaacaaaaacagctcaacgatgccgtcagccgtgaaaataaaaaaatcgaagagcaaaacaaaaaaatcgaagaacaaaaccgcctgcaaaaagaagacaactgcaaaaccgcgcgcctcaaccgccagtttgccgaaaccgcccgcgtggccaaccgcgatgaatttatcaagcgttacgatgccgatatcggcaagttctgcaattag

SEQ ID NO: 12 - DNA, 207 bp, synthetic construct
  source 1..207: mol_type = other DNA
atggatgaagcggtgtttgctgattgggcgttgaaaatctgcctgggcgggctggtggtttttttgggatttatcgtttggaatctgggtaaggaatcgaaagcgggaaaattcggtatgtttgtgctgtttttggtgttggggctgggtgtgttcggctttcttttcaaaaatgtgttaatcgaatttttggtgttgtcgaaataa

SEQ ID NO: 13 - DNA, 699 bp, synthetic construct
  source 1..699: mol_type = other DNA
atggcaaaaaatgcggctgaaagcggcggcatctccagcgcattgaaaaaatacctgattacaggtgttttggtgtggctgccgatagccgtaaccgtgtgggtgatcacctatatcgtttccgcatccgaccggctgttccagctgctgccgctcaaatggcagccggccaacctgataggcttcaacattcccggcttgggcgtgattgtggcggtggccgtgttgtttgccaccggcctgtttgccgccaatatgctgggcaggcagattattgcggtatgggacaacatgctcggccgcattcctgtggtgaaatcgatttattccagcgtaaaaaaagtttcggaatcactgttttccgacaacagccgctcattcaaaacccctgttttagtgccgtttccccagccgggcatctggacgatcgcttttgtgtcgggcagccttgccgatgcagtgggtcgcagcctcgataatggcgaagaatacctttccgtttatgtgccgaccacgcccaaccccaccggcggttattatgtgatggtaaaaaaaagcgatgtgcgcgagctggatatgagcgtggacgatgccttgaaatatgtgatttcgctaggtatggtaatgccggaagaccggtcggcgaaagccctgcccgatgcgcagaagccgtctgaacaccccgcagggcaagaataa

SEQ ID NO: 14 - protein, 107 aa, synthetic construct
MNKLISALFAACVLAACASPNSAGNMAIGDDSAAIKAGRNRMSAELSRSELEQQRRQRTNTAEEIALEEQKRRNKHEGIRDTMRTTSGGLGVINEAIGTIRNLRWGF

SEQ ID NO: 15 - protein, 84 aa, synthetic construct
MFVNNLLIPAFAAVLLLGGCTYADQRFVSSQSQQQQLYENRLAIQKAEREERAERRAERREEMMDEADAVNRAYKGLNGVYLIR

SEQ ID NO: 16 - protein, 136 aa, synthetic construct
MFKRPEELIVAVLAALWVVLAYLAADYLGAPAQTKLLISALTLICTALCFILWQRNLSRAVWPFFLGMLTACWWPYLDWMAVRDILVPGAESSAIIINKPWYAGWTFKTILAAAPVVLGYAVKWKLHSKHKHKAVD

SEQ ID NO: 17 - protein, 200 aa, synthetic construct
MAGWDVWISVVYIAVFFTAAFKAGEFQWLWGSVLLWFGFGLAGARLLPGIWGIIHLAPLYPPHLYIIPASLFFFANHWKKVPEKKMWQAENANGFISLFAVSGMLMGIVSAVLALVVLSRFPAGITPYVLPVLLQLYAFEPAYWFGMQAVIMVVFYLHRKVIAGEAANRFSRLQLQLGFLLALLFQTACTVVLLSDIRYG

SEQ ID NO: 18 - protein, 162 aa, synthetic construct
MKKYLPFLRFVLIACISAFLAWKGQPFVHGNEKAVDLIINVFAILAGFLIAIMTLFSDMRFDEDANWRQIQIREGVQEQRYIKHSMLFYTYLAVLVCVFLVILLAHKEEYKNGQIIFWLERGYLFLACMSIFYSVFLPGNLIKSRKEEFQKLMDKKKPKYPN

SEQ ID NO: 19 - protein, 95 aa, synthetic construct
MNKELLGIVFLPAGVISMCMAALWQMYVVITESYMLNRFKDKQLVWVVAVLFFSFSTAVYWFCPNARKKGILFLLLGAGGAVMYGLARIWLPFGK

SEQ ID NO: 20 - protein, 145 aa, synthetic construct
MKLNRKQAVWAGILLLAFAAVKIAALLWWQSRQPAVTSVAESACNVHAGCTLPNGAQVKFSSKVAAKAPFDITLHNVPAQTAEVFVSFSMRGMDMGFNRYKLLRRPDGSWAAPQIRLPVCVQNRRDYLADIHIGGEVFQVGFTAE

SEQ ID NO: 21 - protein, 91 aa, synthetic construct
MQSTQRKGLVLVSTLAAMAAAFSSGHTEAQSAEMPLSELGCDKLNVLRITQMNGKQQVFARECAAAEAAQAWEQSYGALDSESLIASVIYE

SEQ ID NO: 22 - protein, 148 aa, synthetic construct
MSVYSVAHIIHLFCAIVFVGGVFFETLVLSAIHSKDVPREARRTVEKAIMRRAVRVMPWVVGSVFLSGLTMAHRYAAAFAAPFSAPFNTQLCLKALLAFGVLAHFLIAIIKMRRNTLTAAWSKYIHLAVFCQMVLIVLLAKTMFYIAW

SEQ ID NO: 23 - protein, 86 aa, synthetic construct
MKTTTSTALAALAGALALAGCQAGSAPSAPIAASGQHKVGEGKCGASGKAAEGKCGATSHNGKAAEGKCGAASHSGKSAEGKCGSK

SEQ ID NO: 24 - protein, 147 aa, synthetic construct
MHITRTVYTAALLCLLPAQFAAAQIYTWKGKNGSTSYSDVPHNLKISNASTINVRTRTVTPPPAAQTAESAGEGSLADQQKQLNDAVSRENKKIEEQNKKIEEQNRLQKEDNCKTARLNRQFAETARVANRDEFIKRYDADIGKFCN

SEQ ID NO: 25 - protein, 68 aa, synthetic construct
MDEAVFADWALKICLGGLVVFLGFIVWNLGKESKAGKFGMFVLFLVLGLGVFGFLFKNVLIEFLVLSK

SEQ ID NO: 26 - protein, 232 aa, synthetic construct
MAKNAAESGGISSALKKYLITGVLVWLPIAVTVWVITYIVSASDRLFQLLPLKWQPANLIGFNIPGLGVIVAVAVLFATGLFAANMLGRQIIAVWDNMLGRIPVVKSIYSSVKKVSESLFSDNSRSFKTPVLVPFPQPGIWTIAFVSGSLADAVGRSLDNGEEYLSVYVPTTPNPTGGYYVMVKKSDVRELDMSVDDALKYVISLGMVMPEDRSAKALPDAQKPSEHPAGQE

SEQ ID NO: 27 - DNA, 33 bp, synthetic construct
  source 1..33: mol_type = other DNA
acactctttccctacacgacgctcttccgatct

SEQ ID NO: 28 - DNA, 41 bp, synthetic construct
  source 1..41: mol_type = other DNA
gatcggaagagcggttcagcaggaatgccgagaccgatctc

SEQ ID NO: 29 - DNA, 58 bp, synthetic construct
  source 1..58: mol_type = other DNA
tcgtcggcagcgtcagatgtgtataagagacagcggggatcctctagagtcgacctgc

SEQ ID NO: 30 - DNA, 66 bp, synthetic construct
  source 1..66: mol_type = other DNA
gtctcgtgggctcggagatgtgtataagagacagacactctttccctacacgacgctcttccgatc

SEQ ID NO: 31 - DNA, 20 bp, synthetic construct
  source 1..20: mol_type = other DNA
atcctggctcagattgaacg

SEQ ID NO: 32 - DNA, 20 bp, synthetic construct
  source 1..20: mol_type = other DNA
ccgctttccttctcaaagtg

SEQ ID NO: 33 - DNA, 20 bp, synthetic construct
  source 1..20: mol_type = other DNA
agtatccgtatgccgctcac

SEQ ID NO: 34 - DNA, 20 bp, synthetic construct
  source 1..20: mol_type = other DNA
agctgcaccgaaatatcctg

SEQ ID NO: 35 - DNA, 20 bp, synthetic construct
  source 1..20: mol_type = other DNA
ttaaagaagcggcaggagag

SEQ ID NO: 36 - DNA, 20 bp, synthetic construct
  source 1..20: mol_type = other DNA
acttggtcaagctgggtttg

SEQ ID NO: 37 - DNA, 20 bp, synthetic construct
  source 1..20: mol_type = other DNA
atgcggtaggaaaactgtgg

SEQ ID NO: 38 - DNA, 20 bp, synthetic construct
  source 1..20: mol_type = other DNA
acccacaaagcgtattcctg

SEQ ID NO: 39 - DNA, 20 bp, synthetic construct
  source 1..20: mol_type = other DNA
ccgactaaaggcgagatcag

SEQ ID NO: 40 - DNA, 20 bp, synthetic construct
  source 1..20: mol_type = other DNA
aaagcgcaaattatccatgc

SEQ ID NO: 41 - DNA, 20 bp, synthetic construct
  source 1..20: mol_type = other DNA
cagatggacgttgtgtttgc

SEQ ID NO: 42 - DNA, 20 bp, synthetic construct
  source 1..20: mol_type = other DNA
agggaaaatttcggagaagg

SEQ ID NO: 43 - DNA, 20 bp, synthetic construct
  source 1..20: mol_type = other DNA
attgatctcactggccaacc

SEQ ID NO: 44 - DNA, 20 bp, synthetic construct
  source 1..20: mol_type = other DNA
ggtcgggtgttttattcacg

SEQ ID NO: 45 - DNA, 20 bp, synthetic construct
  source 1..20: mol_type = other DNA
acagcgtaaccagaccttgg

SEQ ID NO: 46 - DNA, 20 bp, synthetic construct
  source 1..20: mol_type = other DNA
agccgggtcttctttattcc

SEQ ID NO: 47 - DNA, 20 bp, synthetic construct
  source 1..20: mol_type = other DNA
tcctgccaatcggattttag

SEQ ID NO: 48 - DNA, 20 bp, synthetic construct
  source 1..20: mol_type = other DNA
tttaatcaggctgggtacgg

SEQ ID NO: 49 - DNA, 20 bp, synthetic construct
  source 1..20: mol_type = other DNA
tggggcagtagctttctttc

SEQ ID NO: 50 - DNA, 20 bp, synthetic construct
  source 1..20: mol_type = other DNA
agggcagcaataccttttcc

SEQ ID NO: 51 - DNA, 20 bp, synthetic construct
  source 1..20: mol_type = other DNA
cgaccataaatggggtgttc

SEQ ID NO: 52 - DNA, 20 bp, synthetic construct
  source 1..20: mol_type = other DNA
atgaacagcggcaggtaatc

SEQ ID NO: 53 - DNA, 20 bp, synthetic construct
  source 1..20: mol_type = other DNA
ttcgaccgctttatcgattc

SEQ ID NO: 54 - DNA, 20 bp, synthetic construct
  source 1..20: mol_type = other DNA
acgggaaacatcgctacatc

SEQ ID NO: 55 - DNA, 20 bp, synthetic construct
  source 1..20: mol_type = other DNA
cataaaaaggcaggccgtag

SEQ ID NO: 56 - DNA, 20 bp, synthetic construct
  source 1..20: mol_type = other DNA
gaccaaaccgtaaccgaatg

SEQ ID NO: 57 - DNA, 20 bp, synthetic construct
  source 1..20: mol_type = other DNA
agctgccttgaaaggtgatg

SEQ ID NO: 58 - DNA, 20 bp, synthetic construct
  source 1..20: mol_type = other DNA
gttacgctcaacagcaccag

SEQ ID NO: 59 - DNA, 20 bp, synthetic construct
  source 1..20: mol_type = other DNA
cgccgattgtcagtagtgtg

SEQ ID NO: 60 - DNA, 20 bp, synthetic construct
  source 1..20: mol_type = other DNA
tagcagctgttgcctgtttc

SEQ ID NO: 61 - DNA, 20 bp, synthetic construct
  source 1..20: mol_type = other DNA
atcggtttgatgctttacgg

SEQ ID NO: 62 - DNA, 20 bp, synthetic construct
  source 1..20: mol_type = other DNA
ttacccgcccttgaataacc

SEQ ID NO: 63 - DNA, 20 bp, synthetic construct
  source 1..20: mol_type = other DNA
caagttcagcaaaggggttg

SEQ ID NO: 64 - DNA, 20 bp, synthetic construct
  source 1..20: mol_type = other DNA
agaatgccgattttctcacc

SEQ ID NO: 65 - DNA, 20 bp, synthetic construct
  source 1..20: mol_type = other DNA
tacgccgataacttttacgc

SEQ ID NO: 66 - DNA, 20 bp, synthetic construct
  source 1..20: mol_type = other DNA
tgccatttaacgaggaaagc

SEQ ID NO: 67 - DNA, 21 bp, synthetic construct
  source 1..21: mol_type = other DNA
ttctcaagctgttcatcatcg

SEQ ID NO: 68 - DNA, 20 bp, synthetic construct
  source 1..20: mol_type = other DNA
tgtagattccgtggtggttg

SEQ ID NO: 69 - DNA, 21 bp, synthetic construct
  source 1..21: mol_type = other DNA
ctttagggaggcaatacatgc

SEQ ID NO: 70 - DNA, 20 bp, synthetic construct
  source 1..20: mol_type = other DNA
tcggccaaacaatggattac

SEQ ID NO: 71 - DNA, 20 bp, synthetic construct
  source 1..20: mol_type = other DNA
ctgaaaacagatgggcaatg

SEQ ID NO: 72 - DNA, 18 bp, synthetic construct
  source 1..18: mol_type = other DNA
aacggtcagcggacaaag

SEQ ID NO: 73 - DNA, 20 bp, synthetic construct
  source 1..20: mol_type = other DNA
gttaaggcatgggcagtttg

SEQ ID NO: 74 - DNA, 20 bp, synthetic construct
  source 1..20: mol_type = other DNA
accgccaatggaacattatc

SEQ ID NO: 75 - DNA, 20 bp, synthetic construct
  source 1..20: mol_type = other DNA
ggagggaaaaccaactgctg

SEQ ID NO: 76 - DNA, 24 bp, synthetic construct
  source 1..24: mol_type = other DNA
tcttagaagcagagatcaacaagg

SEQ ID NO: 77 - DNA, 21 bp, synthetic construct
  source 1..21: mol_type = other DNA
aagcccgaaacttgagaaaag

SEQ ID NO: 78 - DNA, 19 bp, synthetic construct
  source 1..19: mol_type = other DNA
gatttggaaatggccaagg

SEQ ID NO: 79 - DNA, 20 bp, synthetic construct
  source 1..20: mol_type = other DNA
tccgggaataaaggtgtttg

SEQ ID NO: 80 - DNA, 20 bp, synthetic construct
  source 1..20: mol_type = other DNA
aaaccgccgtcaatattctg

SEQ ID NO: 81 - DNA, 19 bp, synthetic construct
  source 1..19: mol_type = other DNA
gattccggcggtattcaag

SEQ ID NO: 82 - DNA, 22 bp, synthetic construct
  source 1..22: mol_type = other DNA
tcctgttggaaataacgtttca

SEQ ID NO: 83 - DNA, 21 bp, synthetic construct
  source 1..21: mol_type = other DNA
tgacggaaatcgtattgcttt

SEQ ID NO: 84 - DNA, 20 bp, synthetic construct
  source 1..20: mol_type = other DNA
actcatttcggccaaacaat

SEQ ID NO: 85 - DNA, 20 bp, synthetic construct
  source 1..20: mol_type = other DNA
ctgaaaacagatgggcaatg

SEQ ID NO: 86 - DNA, 20 bp, synthetic construct
  source 1..20: mol_type = other DNA
gcacaaaaattcaccgaagc

SEQ ID NO: 87 - DNA, 18 bp, synthetic construct
  source 1..18: mol_type = other DNA
cggcgggtatgcagaatc

SEQ ID NO: 88 - DNA, 20 bp, synthetic construct
  source 1..20: mol_type = other DNA
acaaaccggcacagcttact

SEQ ID NO: 89 - DNA, 20 bp, synthetic construct
  source 1..20: mol_type = other DNA
agccattgcgagaaggtttt

SEQ ID NO: 90 - DNA, 20 bp, synthetic construct
  source 1..20: mol_type = other DNA
aagcggcagctttatttgaa

SEQ ID NO: 91 - DNA, 20 bp, synthetic construct
  source 1..20: mol_type = other DNA
tgccgaatacaatcagcagt

SEQ ID NO: 92 - DNA, 20 bp, synthetic construct
  source 1..20: mol_type = other DNA
tccacatcaaccacagcaac

SEQ ID NO: 93 - DNA, 1315 bp, synthetic construct
  source 1..1315: mol_type = other DNA
tttgatagcctccggccgggtgccgaatacaatcagcagttttttagcacgtgcggggggaggggataccgctatgggattaaaagcaaatgaatcgttgaatgttgtcaatgtcagagcactattatcggcgttattgtttttttgcacggcacagcctttcgtattacacgggtagaactttgtcatttaagggatgcagtttatgcatcccttaactcgagggcttgacactttatgcttccggctcgtataatgtgtggatagtgggaggaaagcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagccgtctgaagtttaaacatcgataaaatgccgtccgaagcttgagttcagacggcattttatgacgcgtttccagttgccggccggatgcattctattgctgcttttagggtgtggtgctttgcctacttcagggccgaatcaaaccaaggtgataagtttgcaaaatcaaagccgccaggcggcagtgcctgatgttgctgtggttgatgtggacgagcgggtaatttccacactgtat

SEQ ID NO: 94 - DNA, 20 bp, synthetic construct
  source 1..20: mol_type = other DNA
acgccttcacgaaacagatt

SEQ ID NO: 95 - DNA, 19 bp, synthetic construct
  source 1..19: mol_type = other DNA
aattattctgcgccacacg

(19) United States
(12) Patent Application Publication
(10) Pub. No.: US 2025/0099566 A1
(43) Pub. Date: Mar. 27, 2025

(54) COMPOSITIONS AND METHODS FOR TREATING BACTERIAL DISEASE

(71) Applicant: Arizona Board of Regents on Behalf of the University of Arizona, Tucson, AZ (US)

(72) Inventors: Magdalene So, Tucson, AZ (US); Katherine Rhodes, Tucson, AZ (US); Maria Rendon, Tucson, AZ (US)

(21) Appl. No.: 18/727,485

(22) PCT Filed: Jan. 11, 2023
(86) PCT No.: PCT/US2023/060468

§ 371 Date: Jul. 9, 2024

Related U.S. Application Data
(60) Provisional application No. 63/298,378, filed on Jan. 11, 2022.

(51) Int. Cl. A61K 39/095; A61K 39/00; A61P 31/04; C12N 15/113; C12N 15/86; G01N 33/68

(52) U.S. Cl. CPC A61K 39/095; A61P 31/04; C12N 15/113; C12N 15/86; G01N 33/68; A61K 2039/53; A61K 2039/55555; C12N 2710/10034; C12N 2710/10043; G01N 2333/22

(57) ABSTRACT

The present invention relates to compositions and methods for preventing and/or treating bacterial disease (e.g., disease caused by Neisseria sp. such as gonorrhea). In particular, the present invention provides vaccine compositions and agents targeting Neisseria host interaction genes.

### STATEMENT REGARDING RELATED APPLICATIONS

This application claims priority to U.S. Provisional Application No. 63/298,378, filed Jan. 11, 2022, the entire contents of which are incorporated herein by reference for all purposes.

### STATEMENT REGARDING FEDERALLY SPONSORED RESEARCH OR DEVELOPMENT

This invention was made with government support under Grant No. AI144763 awarded by National Institute of Health. The government has certain rights in the invention.

### REFERENCE TO SEQUENCE LISTING

The contents of the electronic sequence listing (“40080_601_SequenceListin”; Size: 91,000 bytes; and Date of Creation: Jan. 11, 2023) is herein incorporated by reference in its entirety.

### FIELD OF THE INVENTION

The present invention relates to compositions and methods for preventing and/or treating bacterial disease (e.g., disease caused by Neisseria sp. such as gonorrhea). In particular, the present invention provides vaccine compositions and agents targeting Neisseria host interaction genes.

### BACKGROUND

Neisseria gonorrhoeae (Ngo) and Neisseria meningitidis (Nine) are pathogens that cause high impact diseases in humans. Ngo infects the urinary tract and oropharynx of males and females. Ngo causes over 160 million new infections each year, worldwide. There is currently no vaccine against Ngo. Ngo has developed resistance to all antibiotics used for its treatment, leading the NIH, CDC and WHO to place Ngo on their list of “superbugs”. NIH has announced initiatives to accelerate the development of novel antimicrobials and identify new targets for antimicrobials and antibiotics against these superbugs.

Nine colonizes the upper respiratory tract, entering the bloodstream to cause septicemia and crossing the blood-brain barrier to cause meningitis. Crowded living conditions and large migrations encourage the spread of Nine, and are the main cause of epidemics and micro-epidemics around the world. As the CDC does not require the reporting of Nine infections, there is no accurate information on their incidence. Vaccines have significantly reduced the incidence of meningococcal disease in developed countries.

However, they do not cover all Nine serogroups and are unaffordable in poor countries. Nine continues to cause occasional epidemics in parts of Africa and the Middle East.

Improved therapeutic options for treating gonorrhea and/or conditions involving Neisseria gonorrhoeae and Neisseria meningitidis activity are needed.

### SUMMARY OF THE INVENTION

The mechanisms used by human adapted commensal Neisseria to shape and maintain a host niche are poorly defined. These organisms are common members of the mucosal microbiota and share many putative host interaction factors with Neisseria meningitidis and Neisseria gonorrhoeae. Evaluating the role of these shared factors during host carriage provides insight into bacterial mechanisms driving both commensalism and asymptomatic infection across the genus.

Experiments described herein identified host interaction factors required for niche development and maintenance using transposon mutagenesis and screening of Neisseria musculi, a commensal species that persistently and asymptomatically colonizes the oral cavity and gut of laboratory mice. These included homologs of putative virulence factors encoded by Neisseria meningitidis and Neisseria gonorrhoeae, as well as a number of genes of unknown function, conserved in human adapted Neisseria. Validation of a subset of candidate genes confirmed a requirement for a polysaccharide capsule for Nmus survival in the host. The findings provide targets for vaccine and targeted therapeutics.

For example, in some embodiments, provided herein is a vaccine composition, comprising: a) at least a portion (e.g., at least 10, 50, 100, 1000, 2000 nucleotides or the full length) of a plurality (e.g., 1, 2, 3, 4, 5, 6, 7, 8, 9, 10, 11, 12, or all) nucleic acids selected from SEQ ID NOs: 1-13 or at least a portion (e.g., at least 5, 10, 50, 100, 500 amino acids or the full length) of a plurality (e.g., 1, 2, 3, 4, 5, 6, 7, 8, 9, 10, 11, 12, or all) polypeptides selected from SEQ ID NOs: 14-26; and b) a pharmaceutically acceptable carrier.

In some embodiments, the nucleic acid is mRNA, DNA, or cDNA. In some embodiments, the nucleic acid is in a vector (e.g., an adenoviral vector).

The present disclosure is not limited to a particular pharmaceutically acceptable carrier. Examples include, for example, an adjuvant or a lipid.

Further embodiments provide a method of generating an immune response, comprising: administering a vaccine composition described herein to a subject in need thereof. In some embodiments, the immune response generates immunity against infection by a Neisseria sp. (e.g., Neisseria gonorrhoeae (Ngo) or Neisseria meningitidis (Nine)).

Additional embodiments provide the use of a vaccine composition described herein to generate an immune response is a subject or the use of a vaccine composition described herein to prevent infection against a Neisseria sp. in a subject.

Yet other embodiments provide a vaccine composition described herein for use in generating an immune response is a subject or in preventing infection against a Neisseria sp. in a subject.

Also provided is a composition comprising an agent that inhibits the expression or one or more activities of a polypeptide selected SEQ ID NOs: 14-26. The present disclosure is not limited to particular agents. Examples include but are not limited to a nucleic acid (e.g., an siRNA, an antisense nucleic acid, and miRNA, a guide RNA, or a shRNA), a protein, an antibody, or a small molecule.

Still other embodiments provide a method of treating or preventing infection by a Neisseria sp., comprising administering a composition described herein to a subject in need thereof.

Certain embodiments provide the use of a composition described herein to treat or prevent infection by Neisseria sp. in a subject.

In further embodiments, provided is a composition described herein to treat or prevent infection by Neisseria sp. in a subject.

Specific embodiments provide a method of screening a compound, comprising: a) contacting a polypeptide selected from SEQ ID NOs: 14-26 with a test compound; and b) assaying the effect of the test compound on the expression or one or more activities of the polypeptide.

The present disclosure additionally provides a composition, comprising:) at least a portion (e.g., at least 10, 50, 100, 1000, 2000 nucleotides or the full length) of a plurality (e.g., 1, 2, 3, 4, 5, 6, 7, 8, 9, 10, 11, 12, or all) nucleic acids selected from SEQ ID NOs: 1-13 or at least a portion (e.g., at least 5, 10, 50, 100, 500 amino acids or the full length) of a plurality (e.g., 1, 2, 3, 4, 5, 6, 7, 8, 9, 10, 11, 12, or all) polypeptides selected from SEQ ID NOs: 14-26.

Additional embodiments are described herein.

### BRIEF DESCRIPTION OF THE DRAWINGS

FIG. 1A-B. Construction and screening of the N. musculi Tn5 transposon library. A. Genomic DNA was extracted from Neisseria musculi (Nmus) and transposed in vitro using a Tn5EZ-KM kit prior to transformation into Wt Nmus and selection on kanamycin. B. Total bacterial burden recovered from library 1 and 2 cohorts. Data expressed as CFU/ml total sample volume for oral isolates, CFU/gm fecal pellet for gut samples.

FIG. 2A-D. Identification of essential genes during host passage. Volcano plots comparing log 2 Fold Change of Oral Week 8 vs. Inoculum (A.), Gut Week 6 vs. Inoculum (B.) or Gut Week 8 vs. Inoculum (C.) samples to multiple comparison adjusted false discovery rate adjusted P value (q). D. Comparison of significant genes between host essential and housekeeping candidate pools. Genes appearing in both the housekeeping candidate pool, and the host candidate pool were removed from further host-essentiality analysis.

FIG. 3. Function-based classification of candidate gene function. COG category classification of candidate genes identified either as essential to in vitro growth (housekeeping) or significant in host passage samples (Gut, Gut and Oral, Oral).

FIG. 4. Homologs of human adapted Neisseria host interaction factors are required for N. musculi survival in mice. Relative abundance of Nmus encoded Neisseria host interaction factor homologs identified in the in vivo screen Tn5 library host passage. Heatmap depicts relative log₂ fold change of Nmus oral (Week 8), Week 6 gut and Week 8 gut samples in comparison to inoculum.

FIG. 5: Identification of host essential candidate genes within putative prophage regions PHASTER annotation of the Nmus genome identified 11 regions associated with phage or phage like elements. Pink boxes indicate intact prophage regions while orange boxes indicate incomplete prophage regions as designated by PHASTER scoring algorithms. Arrows indicate abundance of insertion mutants after host passage in comparison to the inoculum. Open reading frames passing significance cutoffs after analysis are listed in green boxes for each prophage region.

FIG. 6A-E: N. musculi mutants with a deletion of the capsule cluster regulatory region are uncapsulated. Validation of the candidate genes implicated in capsule biosynthesis and transport. A. Arrangement of the capsule biosynthesis and transport operon of Nmus. Shaded boxes indicate Neisseria genus capsule locus. B-D. Deletion of the capsule locus intergenic region ablates transcription of all capsule biosynthetic and transport genes. cDNA was generated from Wt (B), cps mutant (C) and the complemented strain (D), and primers specific to each gene were used to amplify transcript co-transcribed from the promoter located in the 228 bp intergenic region.

FIG. 7A-B. Loss of capsule significantly reduces N. musculi bacterial burden in the mouse oral cavity but not the gut during persistent colonization. A/J mice were orally inoculated with either Wt, Δ228cps or complemented strains (n=5 per group).

FIG. 8A-C. In vitro fitness analysis of N. musculi. A. Biotradis essentiality analysis of library passage 1 (top panel) and 2 (bottom panel) identified approximately 1100 candidate essential genes. Removal of transposon elements left 968 B. Barplot provides an overview of genes mapping to a subset of 30 pathways. C. Gene concept network plot depicts pathway association of a subset of genes putatively required for in vitro fitness.

FIG. 9A-B. Gene enrichment analysis of host-passage candidates. Candidate genes from host passage samples were analyzed by clusterProfiler analysis using the Nmus Ap2031 KEGG annotation. A. Bar plot depicts pathways whose genes are significantly overrepresented in oral or gut samples compared to inoculum. B. Gene network plot depicts clustering of host enriched pathways to identify functional modules.

FIG. 10. The capsule locus of Neisseria musculi is regulated by the ctrA-A1/csiA intergenic region. cDNA was generated from Wt Nmus total RNA, and primers flanking 5′ and 3′ ends of consecutive genes were used to amplify transcripts transcribed from the 228 bp intergenic region. Top panel: Wt genomic DNA (gDNA) control; middle panel: RT-PCR of wt cDNA; bottom panel: -RT controls.

FIG. 11. Deletion of the capsule locus intergenic sequence does not affect growth rate. Bacteria were cultured from an initial OD600=0.05 in supplemented GCB broth. Optical density was measured for 8 hrs, with no significant differences in growth observed between Wt, Äcps228 mutants and the complemented strain.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “subject” and “patient” refer to any animal, such as a mammal or other animal, for example, a dog, cat, bird, livestock, and preferably a human.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “administration” refers to the act of giving a drug, prodrug, antibody, vaccine, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

The term “antigen” refers to a molecule (e.g., a protein, glycoprotein, lipoprotein, lipid, nucleic acid, or other substance) that is reactive with an antibody specific for a portion of the molecule.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

As used herein, a “vaccine” comprises one or more immunogenic antigens intentionally administered to induce acquired immunity in the recipient (e.g., a subject).

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein the term “epitope” refers to a peptide sequence which elicits an immune response, from either T cells or B cells or antibody.

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. “Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

### DETAILED DESCRIPTION

The microbiota plays a key role in human immune system development and maintenance, nutrient acquisition, and hormone production, among other processes (Maruvada, P., et al., The Human Microbiome and Obesity: Moving beyond Associations. Cell Host Microbe, 2017. 22(5): p. 589-599; Ridaura, V. K., et al., Gut microbiota from twins discordant for obesity modulate metabolism in mice. Science, 2013. 341(6150): p. 1241214; Sommer, F. and F. Backhed, The gut microbiota—masters of host development and physiology. Nat Rev Microbiol, 2013. 11(4): p. 227-38). How microbes/commensals engage the host to shape and maintain their own ecological niche is still not well understood. Dissecting the contribution of individual species to these processes faces considerable hurdles. Most members of the microbiota are not yet culturable, and of those that are, many are genetically intractable (Browne, H. P., et al., Culturing of ‘unculturable’ human microbiota reveals novel taxa and extensive sporulation. Nature, 2016. 533(7604): p. 543-546; Walker, A. W., et al., Phylogeny, culturing, and metagenomics of the human gut microbiota. Trends Microbiol, 2014. 22(5): p. 267-74). The tropism of some species for humans limits the ability of small animal models to accurately recapitulate microbe-human interactions, especially in the context of long-term carriage. To identify the mechanisms used by commensals to interact with their natural host, novel approaches are required.

The Neisseria genus provides this opportunity. This large group of Gram-negative b-Proteobacteria are commonly found in humans and animals (Bennett, J. S., et al., The Genus Neisseria. 4th edition ed. The Prokaryotes—Alphaproteobacteria and Betaproteobacteria, ed. E. Rosenberg. 2014, Berlin Heidelberg: Springer-Verlag). Neisseria sicca, N. subflava, N. flavescens and N. lactamica, among others, inhabit the human oropharynx, establishing their niche early in human development (Sulyanto, R. M., et al., The Predominant Oral Microbiota Is Acquired Early in an Organized Pattern. Sci Rep, 2019. 9(1): p. 10550; Donati, C., et al., Uncovering oral Neisseria tropism and persistence using metagenomic sequencing. Nat Microbiol, 2016. 1(7): p. 16070; Dorey, R. B., et al., The nonpathogenic commensal Neisseria: friends and foes in infectious disease. Curr Opin Infect Dis, 2019. 32(5): p. 490-496). Neisseria spp. have also been identified in diverse wild animals (woodlice: N. perflava, dolphins: N. mucosa, sea lions: N. zalophi, iguanas: N. iguanae, rhesus macaques: N. macacae) and domesticated animals (cattle: N. dentiae, and dogs: N. weaveri) (Liu, G., C. M. Tang, and R. M. Exley, Non-pathogenic Neisseria: members of an abundant, multi-habitat, diverse genus. Microbiology (Reading), 2015. 161(7): p. 1297-1312).

The recent isolation and characterization of Neisseria musculi (Nmus) from a wild-caught mouse opened an avenue of approach for studying commensal colonization (Ma, M., et al., A Natural Mouse Model for Neisseria Colonization. Infect Immun, 2018. 86(5); Weyand, N. J., et al., Isolation and characterization of Neisseria musculi sp. nov., from the wild house mouse. Int J Syst Evol Microbiol, 2016. 66(9): p. 3585-3593; Rhodes, K., M. Ma, and M. So, A Natural Mouse Model for Neisseria Persistent Colonization. Methods Mol Biol, 2019. 1997: p. 403-412). Nmus establishes long-term (at least one year) residence in the oral cavity and gut of susceptible strains of lab mice; the animals are healthy throughout. The Nmus genome has been sequenced and annotated, and, like the other Neisseria spp. examined, the bacterium can be manipulated genetically. The pairing of Nmus with the mouse, its natural host, makes it possible to study commensal Neisseria niche development and maintenance, from the standpoint of both bacterium and host.

Nine and Ngo descended from a commensal Neisseria ancestor and are genetically related to present day commensal Neisseria spp (Quillin, S. J. and H. S. Seifert, Neisseria gonorrhoeae host adaptation and pathogenesis. Nat Rev Microbiol, 2018. 16(4): p. 226-240). This shared ancestry with commensals supports that their tendency to cause persistent asymptomatic infection may be governed by a shared repertoire of host interaction factors. Indeed, of the approximately 177 Ngo and Nine genes previously reported to encode host interaction factors, 69 are conserved in all 19 other Neisseria spp. evaluated (Marri, P. R., et al., Genome sequencing reveals widespread virulence gene exchange among human Neisseria species. PLoS One, 2010. 5(7): p. e11835). While genes unique to Ngo and Nine undoubtedly contribute to pathogenesis, the importance of the shared host interaction factors to infection cannot be discounted. Moreover, these shared host interaction factors may also contribute to the ability of commensal Neisseria to sporadically cause disease.

In experiments described herein, to identify Nmus genes essential for long-term colonization, a library of Tn5 transposon mutants was screened in mice for mutants that failed to persist in the oral cavity and gut. By comparing the inoculum library with those recovered from the two sites at various times after inoculation, a set of Nmus genes that are essential for long-term carriage in these niches were identified. Several Ngo and Nine host interaction genes are on this list, confirming the in vivo importance of these pathogen homologues. Also on the list are genes for the biosynthesis of a polysaccharide capsule, which is widely considered a virulence factor of Nine and other pathogens. In vivo testing of a genetically defined capsule deficient mutant confirmed the essential role this “virulence” factor plays in long-term colonization by Nmus.

Accordingly, provided herein are vaccine compositions and therapeutic agents that target the host interaction factors described herein. In some embodiments, the genes are at least a portion (e.g., at least 10, 50, 100, 1000, 2000 nucleotides or the full length) of a plurality (e.g., 1, 2, 3, 4, 5, 6, 7, 8, 9, 10, 11, 12, or all) nucleic acids selected from SEQ ID NOs: 1-13 or at least a portion (e.g., at least 5, 10, 50, 100, 500 amino acids or the full length) of a plurality (e.g., 1, 2, 3, 4, 5, 6, 7, 8, 9, 10, 11, 12, or all) polypeptides selected from SEQ ID NOs: 14-26. Tables 5 and 6 provide SEQ ID NOs 1-26.

For example, in some embodiments, provided herein is a vaccine composition, comprising: a) at least a portion (e.g., at least 10, 50, 100, 1000, 2000 nucleotides or the full length) of a plurality (e.g., 1, 2, 3, 4, 5, 6, 7, 8, 9, 10, 11, 12, or all) nucleic acids selected from SEQ ID NOs: 1-13 or at least a portion (e.g., at least 5, 10, 50, 100, 500 amino acids or the full length) of a plurality (e.g., 1, 2, 3, 4, 5, 6, 7, 8, 9, 10, 11, 12, or all) polypeptides selected from SEQ ID NOs: 14-26; and b) a pharmaceutically acceptable carrier.

Vaccine compositions can comprise nucleic acids encoding the described genes or polypeptides encoded by the genes. In some embodiments, the nucleic acid is mRNA, DNA, or cDNA. In some embodiments, the nucleic acid is in a vector (e.g., an adenoviral vector).

The present disclosure is not limited to a particular pharmaceutically acceptable carrier. Examples include, for example, an adjuvant or a lipid. Exemplary carriers are described herein.

In embodiments, the present invention provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide described herein.

Polypeptides or peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest is constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest are inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. In order to obtain high expression levels of a transfected gene in a host, the gene can be operatively linked to transcriptional and translational expression control sequences that are functional in the chosen expression host. Recombinant expression vectors may be used to amplify and express DNA encoding the tumor specific neo-antigenic peptides. Recombinant expression vectors are replicable DNA constructs which have synthetic or cDNA-derived DNA fragments encoding a tumor specific neo-antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

The optimum amount of each peptide or nucleic acid to be included in the vaccine composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA or RNA (e.g., siRNA, mRNA) injection include i.d., i.m., s.c, i.p. and i.v. For example, doses of between 1 and 500 mg 50 μg and 1.5 mg, preferably 10 μg to 500 μg, of peptide or DNA or RNA (e.g., siRNA, mRNA) may be given and will depend from the respective peptide or DNA or RNA (e.g., siRNA, mRNA). Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine composition are known to those skilled in the art.

Such embodiments are not limited to a particular type of adjuvant. Generally, adjuvants are any substance whose admixture into the vaccine composition increases or otherwise modifies the immune response to the nucleic acid, polypeptide, or peptide. Carriers are scaffold structures, for example a polypeptide or a polysaccharide, to which the antigenic peptide (e.g., neo-antigenic peptide) is capable of being associated. Optionally, adjuvants are conjugated covalently or non-covalently to the nucleic acids, peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel® vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS).

In some embodiments, the adjuvant is CpG. CpG immuno stimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5 Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5, 6-Dimethylxanthenone-4-Acetic Acid, Journal of Immunology, 190:5216-25 and Kim et al. (2013) Anticancer Flavonoids are Mouse-Selective STING Agonists, 8: 1396-1401). Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′ 5′-OAS and the Pl/eIF2a kinase, also known as the PKR (4-6), as well as RIG-I helicase and MDA5.

Self-replicating RNA vaccines have displayed increased immunogenicity and effectiveness after formulating the RNA in a cationic nanoemulsion based on the MF59 (Novartis) adjuvant (Brito, L. A. et al. A cationic nanoemulsion for the delivery of next-generation RNA vaccines. Mol. Ther. 22, 2118-2129 (2014)). Another effective adjuvant strategy is TriMix, a combination of mRNAs encoding three immune activator proteins: CD70, CD40 ligand (CD40L) and constitutively active TLR4. The type of mRNA carrier and the size of the mRNA-carrier complex have also been shown to modulate the cytokine profile induced by mRNA delivery.

Efficient in vivo mRNA delivery is critical to achieving therapeutic relevance. Exogenous mRNA must penetrate the barrier of the lipid membrane in order to reach the cytoplasm to be translated to functional protein. There are two basic approaches for the delivery of mRNA vaccines that have been described to date. First, loading of mRNA into DCs ex vivo, followed by re-infusion of the transfected cells (Benteyn, D., Heirman, C., Bonehill, A., Thielemans, K. & Breckpot, K. mRNA-based dendritic cell vaccines. Expert Rev. Vaccines 14, 161-176 (2015)) and second, direct parenteral injection of mRNA with or without a carrier.

DCs are the most potent antigen-presenting cells of the immune system. They initiate the adaptive immune response by internalizing and proteolytically processing antigens and presenting them to CD8+ and CD4+ T cells on major histocompatibility complexes (MHCs), namely, MHC class I and MHC class II, respectively. Additionally, DCs may present intact antigen to B cells to provoke an antibody response. DCs are also highly amenable to mRNA transfection. For these reasons, DCs represent an attractive target for transfection by mRNA vaccines, both in vivo and ex vivo.

Cationic lipids and polymers, including dendrimers, have become widely used tools for mRNA administration in the past few years (See e.g., Pardi, Michael J. Hogan, Frederick W. Porter & Drew Weissman Nature Reviews Drug Discovery volume 17, pages 261-279 (2018)). The mRNA field has clearly benefited from the substantial investment in in vivo small interfering RNA (siRNA) administration, where these delivery vehicles have been used for over a decade. Lipid nanoparticles (LNPs) have become one of the most appealing and commonly used mRNA delivery tools. LNPs often consist of four components: an ionizable cationic lipid, which promotes self-assembly into virus-sized (˜100 nm) particles and allows endosomal release of mRNA to the cytoplasm; lipid-linked polyethylene glycol (PEG), which increases the half-life of formulations; cholesterol, a stabilizing agent; and naturally occurring phospholipids, which support lipid bilayer structure. Systemically delivered mRNA-LNP complexes mainly target the liver owing to binding of apolipoprotein E and subsequent receptor-mediated uptake by hepatocytes, and intradermal, intramuscular and subcutaneous administration have been shown to produce prolonged protein expression at the site of the injection.

The present disclosure further provides a method of generating an immune response (e.g., to generates immunity against infection by a Neisseria sp. (e.g., Neisseria gonorrhoeae (Ngo) or Neisseria meningitidis (Nine)) comprising administering a vaccine composition described herein. In some embodiments, the vaccine composition is administered orally, intramuscularly, nasally, or via another delivery mechanism. In some embodiments, one or more (e.g., 1, 2, 3, or more) initial doses of the vaccine are administered over a period of time (e.g., weekly, monthly, every two months, every three months, every four months, every 5 months, every 6 months, or another interval). Following the initial administration, booster doses can be administered as needed (e.g., every 6 months, yearly, or another interval) as needed.

Also provided is a composition comprising an agent that inhibits the expression or one or more activities of a polypeptide selected from SEQ ID NOs: 14-26. The present disclosure is not limited to particular agents. Examples include but are not limited to a nucleic acid (e.g., an siRNA, an antisense nucleic acid, and miRNA, a guide RNA, or a shRNA), a protein, an antibody, or a small molecule.

In some embodiments, the inhibitor is a nucleic acid. Exemplary nucleic acids suitable for inhibiting a gene described herein (e.g., by preventing expression of the gene) include, but are not limited to, antisense nucleic acids, miRNAs, and shRNAs. In some embodiments, nucleic acid therapies are complementary to and hybridize to at least a portion (e.g., at least 5, 8, 10, 11, 12, 13, 14, 15, 16, 17, 18, 19, or 20 nucleotides) of SEQ ID NOs:1-13.

In some embodiments, compositions comprising oligomeric antisense compounds, particularly oligonucleotides are used to modulate the function of nucleic acid molecules encoding the genes described herein, ultimately modulating the amount of the gene expressed. This is accomplished by providing antisense compounds that specifically hybridize with one or more nucleic acids encoding the gene(s). The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of CDK6. In the context of the present disclosure, “modulation” means either an increase (stimulation) or a decrease (inhibition) in the expression of a gene. For example, expression may be inhibited to treat or prevent a metabolic disorder.

In some embodiments, nucleic acids are siRNAs. “RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, SIN3A. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding SIN3A. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been recently shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (.about.35 nucleotides upstream and .about.40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In some embodiments, the present disclosure provides antibodies that inhibit a gene described herein. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, candidate inhibitors are screened for activity.

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present disclosure the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the composition is a gel (e.g., formulated for delivery to a mucosal surface). In some embodiments, the gel coats a product for use in treating or preventing infection by Ngo or Nine (e.g., a condom). In some embodiments, the composition stabilizes the nucleic acid from degradation by enzymes in the mucosa.

In some embodiments, the composition is formulated for delivery to the oropharynx (e.g., as a toothpaste or mouthwash).

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating clinician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

The composition according to the present invention can be co-administered to an individual in need thereof in combination with one or more drugs such as one or more drugs with antibacterial effect. The one or more antibiotics can be selected from the group consisting of Amikacin disulfate salt, Amikacin hydrate, Anisomycin from Streptomyces griseolus, Apramycin sulfate salt, Azithromycin, Blasticidine S hydrochloride, Brefeldin A, Brefeldin A from Penicillium brefeldianum, Butirosin sulfate salt, Butirosin A from Bacillus vitellinus, Chloramphenicol, Chloramphenicol base, Chloramphenicol succinate sodium salt, Chlortetracycline hydrochloride, Chlortetracycline hydrochloride from Streptomyces aureofaciens, Clindamycin 2-phosphate, Clindamycin hydrochloride, Clotrimazole, Cycloheximide from microbial, Demeclocycline hydrochloride, Dibekacin sulfate salt, Dihydrostreptomycin sesquisulfate, Dihydrostreptomycin solution, Doxycycline hyclate, Duramycin from Streptoverticillium cinnamoneus, Emetine dihydrochloride hydrate), Erythromycin, Erythromycin USP, Erythromycin powder, Erythromycin, Temephos, Erythromycin estolate, Erythromycin ethyl succinate, Erythromycin standard solution, Erythromycin stearate, Fusidic acid sodium salt, G 418 disulfate salt, G 418 disulfate salt powder, G 418 disulfate salt solution liquid, Gentamicin solution liquid, Gentamicin solution, Gentamicin sulfate Micromonospora purpurea, Gentamicin sulfate salt, Gentamicin sulfate salt powder USP, Gentamicin-Glutamine solution liquid, Helvolic acid from Cephalosporium caerulens, Hygromycin B Streptomyces hygroscopicus, Hygromycin B Streptomyces hygroscopicus powder, Hygromycin B solution Streptomyces hygroscopicus, Josamycin, Josamycin solution, Kanamycin B sulfate salt, Kanamycin disulfate salt from Streptomyces kanamyceticus, Kanamycin monosulfate from Streptomyces kanamyceticus, Kanamycin monosulfate from Streptomyces kanamyceticus powder USP, Kanamycin solution from Streptomyces kanamyceticus, Kirromycin from Streptomyces collinus, Lincomycin hydrochloride, Lincomycin standard solution, Meclocycline sulfosalicylate salt, Mepartricin, Midecamycin from Streptomyces mycarofaciens, Minocycline hydrochloride crystalline, Neomycin solution, Neomycin trisulfate salt hydrate, Neomycin trisulfate salt hydrate powder, Neomycin trisulfate salt hydrate USP powder, Netilmicin sulfate salt, Nitrofurantoin crystalline, Nourseothricin sulfate, Oleandomycin phosphate salt, Oleandomycin triacetate, Oxytetracycline dihydrate, Oxytetracycline hemicalcium salt, Oxytetracycline hydrochloride, Paromomycin sulfate salt, Puromycin dihydrochloride from Streptomyces alboniger, Rapamycin from Streptomyces hygroscopicus, Ribostamycin sulfate salt, Rifampicin, Rifamycin SV sodium salt, Rosamicin Micromonospora rosaria, Sisomicin sulfate salt, Spectinomycin dihydrochloride hydrate, Spectinomycin dihydrochloride hydrate powder, Spectinomycin dihydrochloride pentahydrate, Spiramycin, Spiramycin from Streptomyces sp., Spiramycin solution, Streptomycin solution, Streptomycin sulfate salt, Streptomycin sulfate salt powder, Tetracycline, Tetracycline hydrochloride, Tetracycline hydrochloride USP, Tetracycline hydrochloride powder, Thiamphenicol, Thiostrepton from Streptomyces azureus, Tobramycin, Tobramycin sulfate salt, Tunicamycin A₁ homolog, Tunicamycin C₂ homolog, Tunicamycin Streptomyces sp., Tylosin solution, Tylosin tartrate, Viomycin sulfate salt, Virginiamycin M₁, (S)-(+)-Camptothecin, 10-Deacetylbaccatin III from Taxus baccata, 5-Azacytidine, 7-Aminoactinomycin D, 8-Quinolinol crystalline, 8-Quinolinol hemisulfate salt crystalline, 9-Dihydro-13-acetylbaccatin III from Taxus canadensis, Aclarubicin, Aclarubicin hydrochloride, Actinomycin D from Streptomyces sp., Actinomycin I from Streptomyces antibioticus, Actinomycin V from Streptomyces antibioticus, Aphidicolin Nigrospora sphaerica, Bafilomycin A1 from Streptomyces griseus, Bleomycin sulfate from Streptomyces verticillus, Capreomycin sulfate from Streptomyces capreolus, Chromomycin A₃ Streptomyces griseus, Cinoxacin, Ciprofloxacin BioChemika, cis-Diammineplatinum(II) dichloride, Coumermycin A1, Cytochalasin B Helminthosporium dematioideum, Cytochalasin D Zygosporium mansonii, Dacarbazine, Daunorubicin hydrochloride, Daunorubicin hydrochloride USP, Distamycin A hydrochloride from Streptomyces distallicus, Doxorubicin hydrochloride, Echinomycin, Echinomycin BioChemika, Enrofloxacin BioChemika, Etoposide, Etoposide solid, Flumequine, Formycin, Fumagillin from Aspergillus fumigatus, Ganciclovir, Gliotoxin from Gliocladium fimbriatum, Lomefloxacin hydrochloride, Metronidazole purum, Mithramycin A from Streptomyces plicatus, Mitomycin C Streptomyces caespitosus, Nalidixic acid, Nalidixic acid sodium salt, Nalidixic acid sodium salt powder, Netropsin dihydrochloride hydrate, Nitrofurantoin, Nogalamycin from Streptomyces nogalater, Nonactin from Streptomyces tsusimaensis, Novobiocin sodium salt, Ofloxacin, Oxolinic acid, Paclitaxel from Taxus yannanensis, Paclitaxel from Taxus brevifolia, Phenazine methosulfate, Phleomycin Streptomyces verticillus, Pipemidic acid, Rebeccamycin from Saccharothrix aerocolonigenes, Sinefungin, Streptonigrin from Streptomyces flocculus, Streptozocin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Trimethoprim, Trimethoprim lactate salt, Tubercidin from Streptomyces tubercidicus, 5-Azacytidine, Cordycepin, Formycin A, (+)-6-Aminopenicillanic acid, 7-Aminodesacetoxycephalosporanic acid, Amoxicillin, Ampicillin, Ampicillin sodium salt, Ampicillin trihydrate, Ampicillin trihydrate USP, Azlocillin sodium salt, Bacitracin Bacillus licheniformis, Bacitracin zinc salt Bacillus licheniformis, Carbenicillin disodium salt, Cefaclor, Cefamandole lithium salt, Cefamandole nafate, Cefamandole sodium salt, Cefazolin sodium salt, Cefinetazole sodium salt, Cefoperazone sodium salt, Cefotaxime sodium salt, Cefsulodin sodium salt, Cefsulodin sodium salt hydrate, Ceftriaxone sodium salt, Cephalexin hydrate, Cephalosporin C zinc salt, Cephalothin sodium salt, Cephapirin sodium salt, Cephradine, Cloxacillin sodium salt, Cloxacillin sodium salt monohydrate, D-~( )-Penicillamine hydrochloride, D-Cycloserine microbial, D-Cycloserine powder, Dicloxacillin sodium salt monohydrate, D-Penicillamine, Econazole nitrate salt, Ethambutol dihydrochloride, Lysostaphin from Staphylococcus staphylolyticus, Moxalactam sodium salt, Nafcillin sodium salt monohydrate, Nikkomycin, Nikkomycin Z Streptomyces tendae, Nitrofurantoin crystalline, Oxacillin sodium salt, Penicillic acid powder, Penicillin G potassium salt, Penicillin G potassium salt powder, Penicillin G potassium salt, Penicillin G sodium salt hydrate powder, Penicillin G sodium salt powder, Penicillin G sodium salt, Phenethicillin potassium salt, Phenoxymethylpenicillinic acid potassium salt, Phosphomycin disodium salt, Pipemidic acid, Piperacillin sodium salt, Ristomycin monosulfate, Vancomycin hydrochloride from Streptomyces orientalis, 2-Mercaptopyridine N-oxide sodium salt, 4-Bromocalcimycin A23187 BioChemika, Alamethicin Trichoderma viride, Amphotericin B Streptomyces sp., Amphotericin B preparation, Calcimycin A23187, Calcimycin A23187 hemi(calcium-magnesium) salt, Calcimycin A23187 hemicalcium salt, Calcimycin A23187 hemimagnesium salt, Chlorhexidine diacetate salt monohydrate, Chlorhexidine diacetate salt hydrate, Chlorhexidine digluconate, Clotrimazole, Colistin sodium methanesulfonate, Colistin sodium methanesulfonate from Bacillus colistinus, Colistin sulfate salt, Econazole nitrate salt, Hydrocortisone 21-acetate, Filipin complex Streptomyces filipinensis, Gliotoxin from Gliocladium fimbriatum, Gramicidin A from Bacillus brevis, Gramicidin C from Bacillus brevis, Gramicidin from Bacillus aneurinolyticus (Bacillus brevis), lonomycin calcium salt Streptomyces conglobatus, Lasalocid A sodium salt, Lonomycin A sodium salt from Streptomyces ribosidificus, Monensin sodium salt, N-(6-Aminohexyl)-5-chloro-1-naphthalenesulfonamide hydrochloride, Narasin from Streptomyces auriofaciens, Nigericin sodium salt from Streptomyces hygroscopicus, Nisin from Streptococcus lactis, Nonactin from Streptomyces sp., Nystatin, Nystatin powder, Phenazine methosulfate, Pimaricin, Pimaricin from Streptomyces chattanoogensis, Polymyxin B solution, Polymyxin B sulfate salt, DL-Penicillamine acetone adduct hydrochloride monohydrate, Polymyxin B sulfate salt powder USP, Praziquantel, Salinomycin from Streptomyces albus, Salinomycin from Streptomyces albus, Surfactin from Bacillus subtilis, Valinomycin, (+)-Usnic acid from Usnea dasypoga, (±)-Miconazole nitrate salt, (S)-(+)-Camptothecin, 1-Deoxymannojirimycin hydrochloride, 1-Deoxynojirimycin hydrochloride, 2-Heptyl-4-hydroxyquinoline N-oxide, Cordycepin, 1,10-Phenanthroline hydrochloride monohydrate puriss, 6-Diazo-5-oxo-L-norleucine, 8-Quinolinol crystalline, 8-Quinolinol hemisulfate salt, Antimycin A from Streptomyces sp., Antimycin A₁, Antimycin A₂, Antimycin A₃, Antipain, Ascomycin, Azaserine, Bafilomycin A1 from Streptomyces griseus, Bafilomycin B1 from Streptomyces species, Cerulenin BioChemika, Chloroquine diphosphate salt, Cinoxacin, Ciprofloxacin, Mevastatin BioChemika, Concanamycin A, Concanamycin A Streptomyces sp, Concanamycin C from Streptomyces species, Coumermycin A1, Cyclosporin A from Tolypocladium inflatum, Cyclosporin A, Econazole nitrate salt, Enrofloxacin, Etoposide, Flumequine, Formycin A, Furazolidone, Fusaric acid from Gibberella fujikuroi, Geldanamycin from Streptomyces hygroscopicus, Gliotoxin from Gliocladium fimbriatum, Gramicidin A from Bacillus brevis, Gramicidin C from Bacillus brevis, Gramicidin from Bacillus aneurinolyticus (Bacillus brevis), Gramicidin from Bacillus brevis, Herbimycin A from Streptomyces hygroscopicus, Indomethacin, Irgasan, Lomefloxacin hydrochloride, Mycophenolic acid powder, Myxothiazol BioChemika, N-(6-Aminohexyl)-5-chloro-1-naphthalenesulfonamide hydrochloride, Nalidixic acid, Netropsin dihydrochloride hydrate, Niclosamide, Nikkomycin BioChemika, Nikkomycin Z Streptomyces tendae, N-Methyl-1-deoxynojirimycin, Nogalamycin from Streptomyces nogalater, Nonactin E80% from Streptomyces tsusimaensis, Nonactin from Streptomyces sp., Novobiocin sodium salt, Ofloxacin, Oleandomycin triacetate, Oligomycin Streptomyces diastatochromogenes, Oligomycin A, Oligomycin B, Oligomycin C, Oligomycin Streptomyces diastatochromogenes, Oxolinic acid, Piericidin A from Streptomyces mobaraensis, Pipemidic acid, Radicicol from Diheterospora chlamydosporia solid, Rapamycin from Streptomyces hygroscopicus, Rebeccamycin from Saccharothrix aerocolonigenes, Sinefungin, Staurosporine Streptomyces sp., Stigmatellin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Triacsin C from Streptomyces sp., Trimethoprim, Trimethoprim lactate salt, Vineomycin A₁ from Streptomyces albogriseolus subsp., Tectorigenin, and Paracelsin Trichoderma reesei.

In a further embodiment the present invention relates to a kit of parts comprising the composition according to the present invention. The kit of parts comprises at least one additional component, such as instructions for use, and/or one or more drugs for coadministration.

The present disclosure further provides drug screening methods. Specific embodiments provide a method of screening a compound, comprising: a) contacting a polypeptide selected from SEQ ID NOs: 14-26 with a test compound; and b) assaying the effect of the test compound on the expression or one or more activities of the polypeptide.

### EXPERIMENTAL

### Materials and Methods

### Bacterial Culture

Nmus strain AP2365 was used for all experiments in this study. Bacteria were struck for single colony isolation on GCB plates containing Kellogg's Supplement I & II and Rifampicin (Rif) (40 g/L) for maintenance, and Kanamycin (Km) (50 g/L) for selection of Tn5-containing clones. All Neisseria cells were grown at 37° C., 5% CO₂.

### DNA Extraction

18-hour lawns of Nmus were collected into TES buffer (50 mM Tris pH 8, 20 mM EDTA, 50 mM NaCl) with 1% SDS and thoroughly resuspended by vortexing (Yi, K., D. S. Stephens, and I. Stojiljkovic, Development and evaluation of an improved mouse model of meningococcal colonization. Infect Immun, 2003. 71(4): p. 1849-55). DNA was extracted from the suspension using two rounds of 25:24:1 phenol/chloroform/isoamyl alcohol extraction followed by ethanol precipitation in sodium acetate (0.3M). RNA was removed with RNAseA (Qiagen) and DNA integrity was confirmed by gel electrophoresis.

### Construction of Tn5 Library

DNA purified from Nmus was modified using the EZ-TN5Km2 transposon kit (Lucigen) with modifications to the manufacturer's instructions as reported in Remelle 2014. 1.5 ug of DNA was incubated 2 hours at 37° C. with 0.15 pmole of EZ-TN5::Km2 transposon, 1.5 units of Tn5 transposase and 1× reaction buffer (0.05 M Tris-acetate pH7.5, 0.15M potassium acetate, 10 mM magnesium acetate, 4 mM spermidine). The reaction was stopped with 0.1% SDS and heated for 20 minutes at 72° C. prior to column purification (Clean and Concentrate kit, Zymo). 1 unit of T4 DNA polymerase (Thermofisher) and 2 nmol of DNTPs (Thermofisher) were added to the heat-killed reactions and incubated another 20 minutes at 11° C. to repair transposon-DNA junctions prior to column purification (Clean and concentrate kit, Zymo). Samples were eluted in Tris (5 mM, pH 8), and stored at −20° C. until transformation. This procedure was repeated three times.

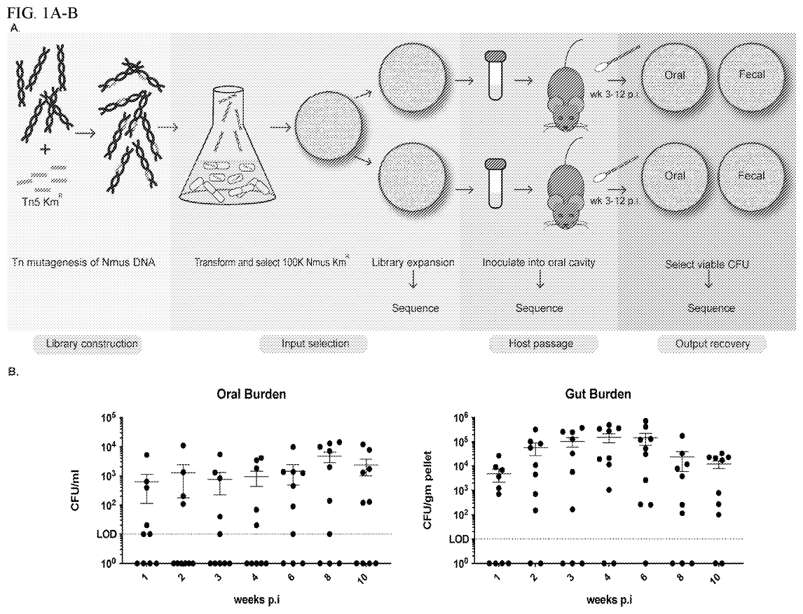

For transformation, 18-hour lawns of Nmus were collected in pre-warmed GCB with 10 mM MgSO₄ and incubated 30 minutes with 1 mg of the in vitro-transposed DNA, spotted on a GCB plate and incubated an additional 8 hours at 37° C. 5% CO₂. Spots were then harvested and selected on GCB agar containing Km (50 g/L) for 48 hours 37° C. 5% CO₂. This procedure was repeated 3 times to collect approximately 100,000 Km^{R} CFUs. Each plate was replicated 5 times onto supplemented GCB agar containing Km (50 g/L) and incubated until colonies were visible (˜24-36 hours). Cells from replicate plates were pooled into single tubes containing 5 ml of GC broth with 20% glycerol to create single library copies, and frozen at −80° C. A summary of the transposon library construction and screening approach is found in FIG. 1.

### Animal Inoculation and Sample Collection

3 days prior to inoculation of the mutant library into mice, one replicate of the library was thawed and plated onto supplemented GCB agar containing Km (50 g/L). After a 36-hour incubation, each plate was replicated onto a fresh agar plate, to ensure only viable bacteria were collected for inoculation, and the plate was incubated another 36 hours. On the day of inoculation, the replicated plates were collected in Dulbecco's PBS, thoroughly vortexed and the OD₆₀₀ was measured in a spectrophotometer. An aliquot of the stock was corrected using fresh sterile DPBS to an OD₆₀₀=2 or OD₆₀₀=4 for inoculation; the remainder was processed for DNA extraction. The inoculum was introduced orally into 5 CAST/EiJ mice pre-screened for endogenous Neisseria, following established procedures. An aliquot of the inoculum was plated on supplemented GCB agar to establish the dose, and the remainder was processed for DNA extraction. Every week for 16 weeks post-inoculation, the mice were cultured for CFU enumeration. Oral swabs were collected as previously described. For each mouse, fecal samples were collected over a 30-minute period in a pre-sterilized canister lined with a sterile paper towel. Pellets from each mouse were collected in a single tube, weighed, then suspended in unsupplemented GC broth with 20% glycerol. A 100 ul aliquot of each sample was plated on supplemented GCB agar containing Rif (40 g/L) to assess bacterial burden; the remainder was banked at −80° C. To prepare samples for sequencing, the remainder of the sample was plated on supplemented GCB agar containing Rif (40 g/L) and Km (50 g/L) to select for Tn5-containing bacteria from each timepoint. The colonies on Rif/Kan plates were pooled by dose, cohort, and experiment, and DNA from each pool was extracted. This process was repeated using a second library replicate in 5 additional CAST/EiJ mice.

For validation of Tn5 mutants, Wt, Δcps228 and complemented strains were harvested in PBS as 18-hour lawns, and adjusted to an OD₆₀₀=2 prior to oral inoculation in 6-8 week old AJ mice according to established procedures. Oral swabs and fecal pellets were collected each week for 8 weeks in GC broth with 20% glycerol for storage at −80° C., and aliquots were plated on supplemented GCB+Rif 40 g/L. CFUs were enumerated, and bacterial burden normalized to total sample volume or CFU/gm fecal pellet for each strain.

### Next Generation Sequencing

Sequencing was performed at the University of Arizona Genetics core following previously described methods (cCarthy, A. J., R. A. Stabler, and P. W. Taylor, Genome-Wide Identification by Transposon Insertion Sequencing of Escherichia coli K1 Genes Essential for In Vitro Growth, Gastrointestinal Colonizing Capacity, and Survival in Serum. J Bacteriol, 2018. 200(7); Remmele, C. W., et al., Transcriptional landscape and essential genes of Neisseria gonorrhoeae. Nucleic Acids Res, 2014. 42(16): p. 10579-95). Briefly, 2 μg of DNA was sheared to 500 bp by ultrasonication on a Covaris S2. Samples were end repaired, A tailed and ligated to adaptors Ind_Ad_T and Ind_Ad_B by NEBnext DNA library prep kit. PCR enrichment of TN5 junctions was performed with primers Tn-FO and Adapt-RO using Kapa HotStart HiFi(Roche) using thermocycling conditions as follows; 95° C. for 5 min, 94° C. for 45 sec, 56° C. for 1 min, 72° C. for 1 min for 22 cycles, and a final extension at 72° C. for 10 minutes. Samples were purified using a MagBio HighPrep PCR cleanup kit (MagBio Genomics) and amplicons from 100 to 600 bp were captured using a 2% agarose cassette on a Blue Pippin instrument (Sage Science). Amplicons were indexed using a Nextera V2 dual index kit (Illumina) and purified by MagBio HighPrep PCR cleanup. Purified, indexed Amplicons were then quantified on a Qbit fluorometer, and sequenced on an Illumina MiSeq instrument as 75 bp paired end reads. Sequencing was repeated on these libraries in three separate runs to generate enough reads for analysis, and the reads were merged by sample after initial demultiplexing and QC.

### Transposon Insertion Sequencing Analysis

Reads were filtered by quality and the presence of the Tn5 sequence on the forward mate read. Illumina adaptor and Tn5 leader sequences were trimmed from each read of the pair, and reads lacking Tn5 sequences were discarded. The resulting reads were aligned to the Nmus reference genome, (accession CP06414.1 and CP06415.1, strain NW831/AP2031) with BWA-MEM (Li, H. and R. Durbin, Fast and accurate short read alignment with Burrows-Wheeler transform. Bioinformatics, 2009. 25(14): p. 1754-60; Thapa, E., et al., Complete Genome Sequence of Neisseria musculi Using Illumina and PacBio Sequencing. Microbiol Resour Announc, 2021. 10(23): p. e0045221). The resulting files were further analyzed using scripts from the BioTradis pipeline, implemented in a conda environment (Barquist, L., et al., The TraDIS toolkit: sequencing and analysis for dense transposon mutant libraries. Bioinformatics, 2016. 32(7): p. 1109-11). Insertion plots were generated as described using the tradis_plot script. To remove multimapping reads, reads with a mapping quality less than 30 were discarded. Gene insertions, i.e., genes interrupted by Tn5, were assigned using tradis_gene_insert_sites script, and insertions in the 3′ 10% of the open reading frame were ignored. Analysis was performed on samples merged by replicate, time point and sample site.

Genes required for in vitro growth fitness were identified using tradis_essentiality.R with passage samples, while conditional essentiality was determined using tradis_comparison.R, comparing inoculum to output samples. An open reading frame was classified as a host essential candidate gene using a cutoff of log 2-fold change >[1] output vs. inoculum, and false discovery rate adjusted p value (q)<0.05. Repetitive transposase elements were removed from the analysis to preclude false positive assignment of essentiality, as these are present in multiple copies in the genome and as such any reads originating from these sites would have been marked as multi-mapping. Genes involved in vitro fitness (housekeeping) were identified as ones appearing as essential in both replicates of the passage sample after essentiality.R analysis. Genes appearing in the housekeeping gene pool were ignored in further analysis of host essential genes. To identify pathways involved in host interaction, pathway enrichment analysis of fold-change ranked significant hits with the enrichKEGG tool from clusterProfiler Bioconductor package implemented in R (Yu, G., et al., clusterProfiler: an R package for comparing biological themes among gene clusters. OMICS, 2012. 16(5): p. 284-7). To expand the number of genes mapped to pathways, analyses were conducted using a minimum gene set size of 1, and a Benjamin-Hochberg adjusted p value cutoff of 0.1. Clustering of mapped pathways was performed using the pairwise term similarity and emap plot functions of clusterProfiler. The Nmus functional annotation hosted on KEGG was used as the reference for these analyses (Kanehisa, M. and S. Goto, KEGG: kyoto encyclopedia of genes and genomes. Nucleic Acids Res, 2000. 28(1): p. 27-30). COG assignments were made using eggnog-mapper V2 with protein sequences from the AP2031 reference (Cantalapiedra, C. P., et al., eggNOG-mapper v2: Functional Annotation, Orthology Assignments, and Domain Prediction at the Metagenomic Scale. Mol Biol Evol, 2021). Phage annotation was performed using the PHASTER server on the Nmus reference genome (Arndt, D., et al., PHASTER: a better, faster version of the PHAST phage search tool. Nucleic Acids Res, 2016. 44(W1): p. W16-21; Zhou, Y., et al., PHAST: a fast phage search tool. Nucleic Acids Res, 2011. 39(Web Server issue): p. W347-52).

### Validation of Capsule Mutants

The noncoding 228 bp DNA segment between the capsular synthesis and transport regions in Nmus was deleted and replaced with a Km cassette. A DNA fragment containing a neomycin phosphotransferase gene and sequences flanking csiA/A1 and ctrA were ligated into pGEMT (Promega) and the ligation product was confirmed by restriction digestion and sequencing. The recombinant plasmid was transformed into Wt Nmus AP2365 and transformants were selected on supplemented GCB agar containing Km (50 ug/ml). Kanamycin resistant mutants were confirmed by Sanger sequencing of PCR amplicons generated by primers IM059 and IM060. To construct the complemented strain, the intergenic region of one capsule nonproducing mutant, Δcps228, was transformed with a PCR product generated from Wt DNA using primers IM133 and IM134.

### Analysis of Cps Transcripts

Wt Nmus AP2365, capsule mutant and complement strains were grown to mid-log phase in supplemented GC broth at 37° C. 5% CO₂ without shaking. Total RNA was extracted using TriZol (Invitrogen) according to the manufacturers recommendations, and treated with DNA-free(ambion) to remove chromosomal DNA. RNA was quantified using a Nanodrop (Thermo scientific), and 1 μg of RNA was used in first strand synthesis using M-MLV reverse transcriptase (Promega) according to the manufacturer's instructions. PCR to characterize transcripts was conducted using GoTaq green master mix (Promega) with primers to amplify transport, translocation and capsule synthesis transcripts and junctions between each putative transcript (Table 1). 16S and gDNA controls were amplified from total RNA.

### Growth Curves

Wt AP2365, capsule mutant and complemented strains were harvested from 16 hour lawns grown on supplemented GCB agar and adjusted to an OD₆₀₀=0.05 in 2 ml GC broth with 1% Kelloggs supplement 1 and 0.1% supplement II in 50 mm dishes. Samples were incubated at 37° C. 5% CO2 for 8 hours, and OD₆₀₀ was measured every 2 hours on a Beckman coulter spectrophotometer. The absorbance at each time point was normalized to the density of that sample at t=0.

### Capsule Extraction and Staining

To examine capsule production, 16 hour lawns of Wt Nmus and capsule mutant strains, Ngo MS11 and Nine FAM18 strains were harvested in PBS, and adjusted to an OD600=1.0. Cell suspensions were incubated at 55° C. for 30 minutes, pelleted and supernates were transferred to a Amicon Ultra 10k NNWL centrifugal filter device (Millipore) and concentrated 10×. 30 ul of concentrate was run on a 7.5% SDS PAGE gel and stained overnight with 0.125% Alcian blue. The gel was destained in 40% ethanol 5% glacial acetic acid for 4 hours, and imaged on a LiCor Odyssey instrument.

### Statistical Analysis

Statistical analysis of transposon insertion data was completed using methods implemented by the BioTradis tool set. Gene enrichment statistical analysis was conducted in R using the clusterProfiler package. Multiple testing was corrected using the Benjamin Hochberg correction for both BioTradis and clusterProfiler analyses. Mouse bacterial burdens were tested for normality using the Shaprio-Wilkes and Kolmogorov-Smirnov tests, and were found to be non-parametric. Bacterial burdens were then analysed by the Kruskal-Wallace test followed by Dunn's comparison to wild type controls to determine significance. All in vitro experiments were conducted in at least biological and technical triplicate, and analyzed using GraphPad Prism V9.

### Example I

Screening of a Library of Tn5 N. musculi Mutants in Mice for Genes Essential for Long Term Colonization

A library of Tn5 mutants of Nmus in the lab mouse was constructed and analyzed to identify mutants in the inoculum that were absent or underrepresented in oral cavity and gut samples at various times post-inoculation. Each of 2 replicates of this library was inoculated into 5 CAST/EiJ mice (approximately 1×10\hat{ }7 mutants per inoculum) (FIG. 1A,B). Nmus burden in the oral cavity and gut were determined weekly for 10 weeks. At week 4, Nmus CFUs were recovered in large numbers from the oral cavity and gut of 50% and 80% of the mice, respectively. By 6 and 8 weeks, Nmus was recovered from 70% of the mice, and viable counts were higher than those from week 4. The number of mice colonized in the oral cavity by Nmus increased over time, while Nmus CFUs recovered from fecal samples remained relatively stable between 4 and 6 weeks.

Nmus DNA was isolated from the inoculum generated from each library as well as from the initial culture from which the inoculum was derived. Tn5 junction enrichment and high throughput sequencing of Tn5 insertion sites was performed at the University of Arizona Next Generation Sequencing facility. A total of ˜62 million Tn5-directed reads were synthesized from all samples; over 90% of mate 1 reads contained the Tn5 adaptor. An average of 75% of the Tn5-containing read pairs in each sample mapped to the Nmus chromosome and plasmid. These reads were derived from 227586 Tn5 insertions in open reading frames in the library 1 inoculum and 244356 in the library 2 inoculum. This represents a transposon insertion every 12-13 bp, allowing for discrimination of essential genes after passage through the mouse. Identification and analysis of Tn5 insertions was performed using tools from the BioTradis pipeline.

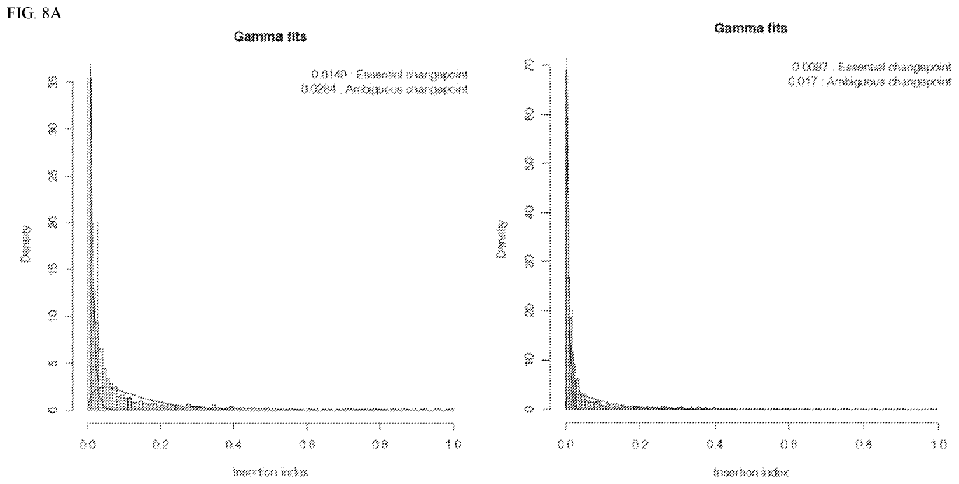

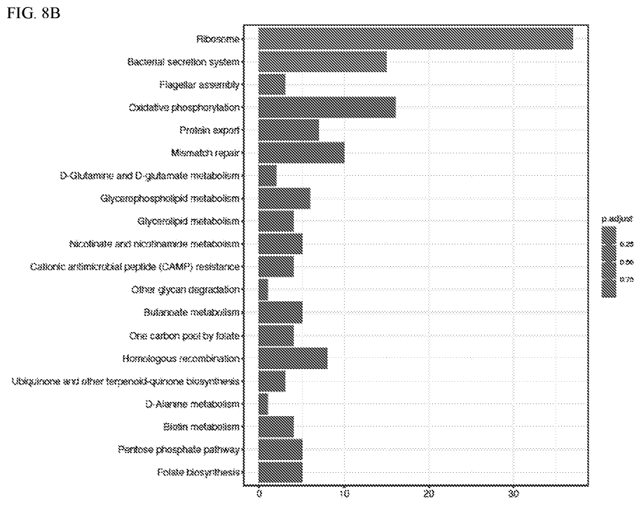

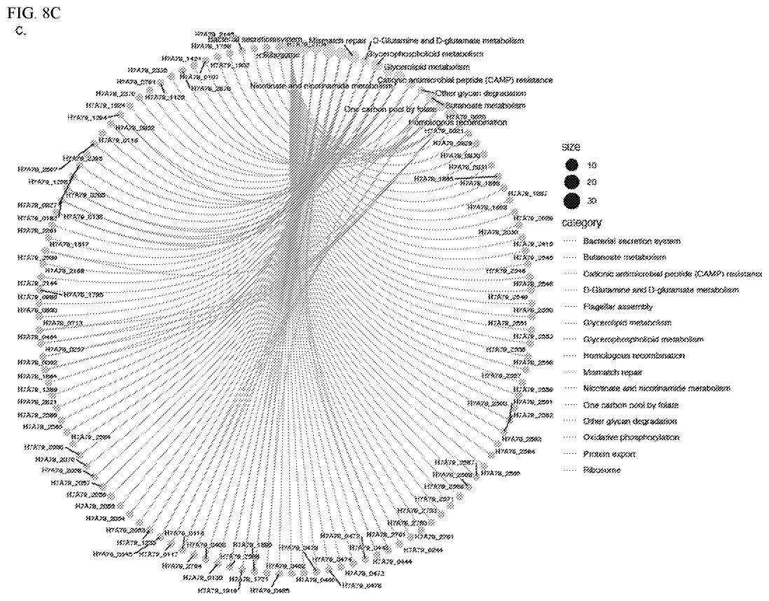

To identify housekeeping genes, the BioTradis essentiality.R tool was used. Approximately ˜1100 candidates met the criteria for essential for in vitro growth with agreement between libraries, based on the distribution of all gene insertion indexes within the sample. (FIG. 8A). These genes were further curated to remove repetitive transposable elements; this avoids genes being erroneously classified as essential due to a lack of insertions as multimapping reads were discarded during the analysis step. The curation yielded a final set of 968 housekeeping genes that are putatively required for growth. Annotation and gene set enrichment analysis revealed their functions are predominately associated with central and secondary metabolism, cell envelope biosynthesis, cell division and nutrient acquisition and transport (FIG. 8B-C).

### Example II

Identification of N. musculi Genes Essential for Long Term Colonization

To focus on genes important for persistence in the mouse, gut samples from week 6 and gut and oral samples from week 8 were further analyzed. Week 6 gut samples contained an average of ˜1.8×10\hat{ }5 Nmus CFU/gm fecal pellet; week 8 gut and oral samples contained an average ˜2.9×10\hat{ }4 CFU/gm fecal pellet and ˜5.0×10\hat{ }4 CFU, in libraries 1 and 2. Bacteria recovered from these samples were pooled by library replicate, dose, time point and sample site, and DNA was extracted and sent for Tn5 insertion sequencing.

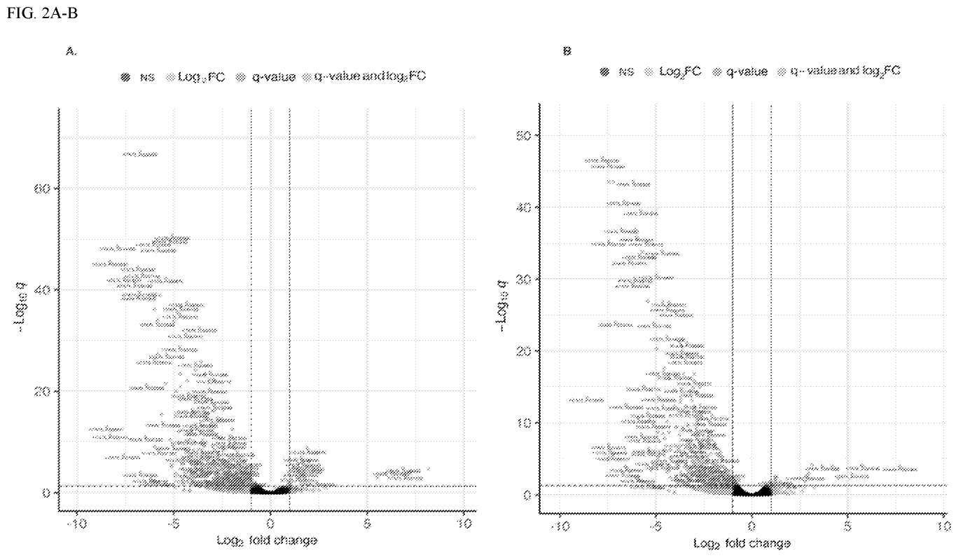

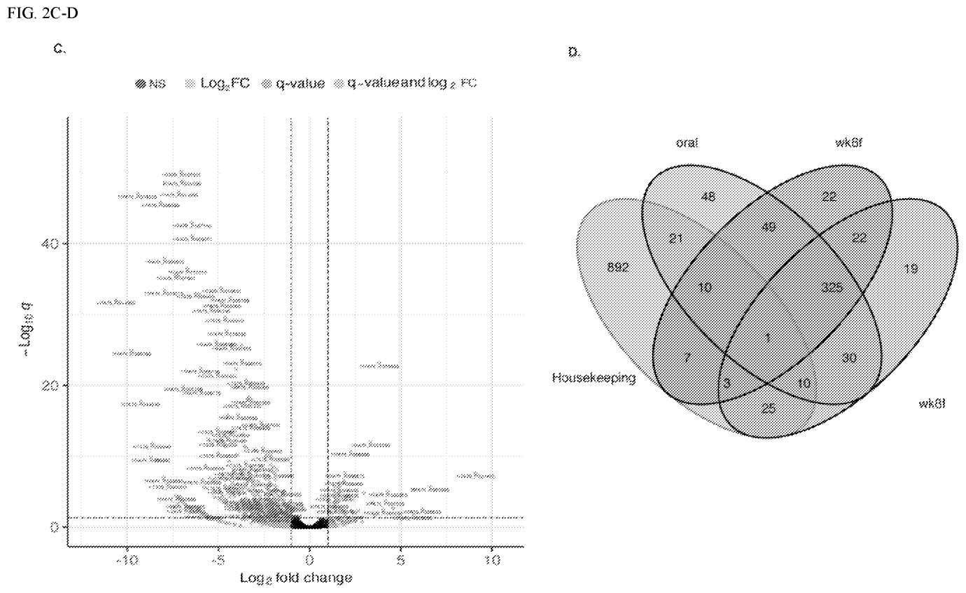

To identify host essential genes, defined as insertion mutants in the inoculum underrepresented in the mouse-passed samples, sequences from oral and gut samples were compared to those from the inoculum, using the BioTradis comparison.R script. This approach identified a total of 592 open reading frames (ORFs) which were underrepresented in the mouse-passaged samples compare to the inoculum (q<0.05 and an absolute Log₂ Fold Change of >1, (FIG. 2A-C). Seventy-seven ORFs from week 6 and 8 samples were also present in the housekeeping gene pool. These were removed from further analysis, as were repetitive transposable elements (FIG. 2D).

Of the remaining 515 candidates, 325 were essential in all three host output samples, 79 were essential in the oral cavity and one gut sample, 48 were unique to the oral cavity, and 64 were unique to the gut (FIG. 2D). On average, 70% of these genes displayed an absolute fold change of 2 compared to the inoculum.

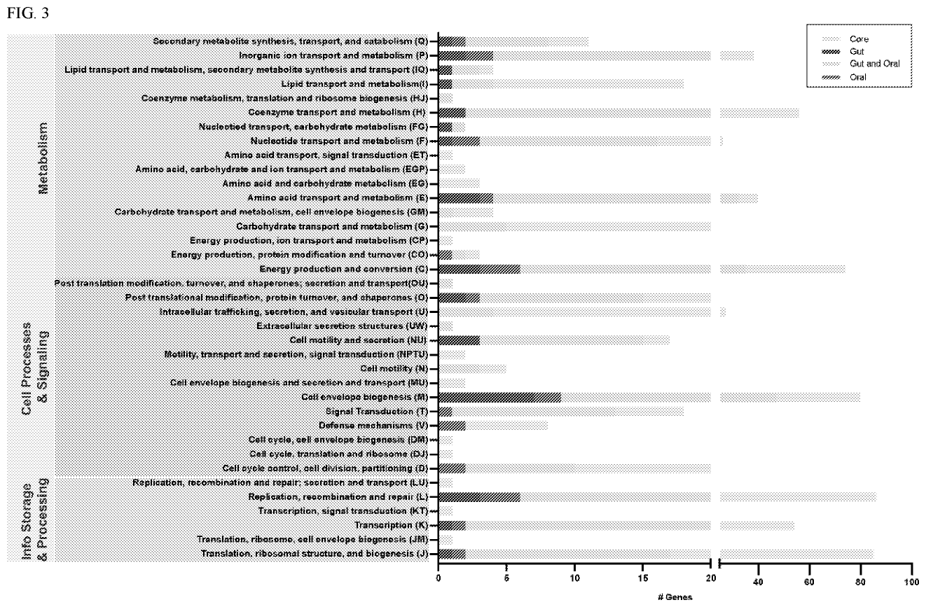

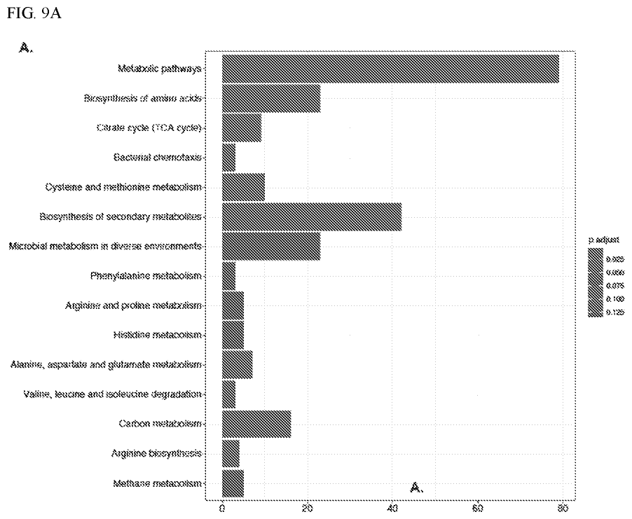

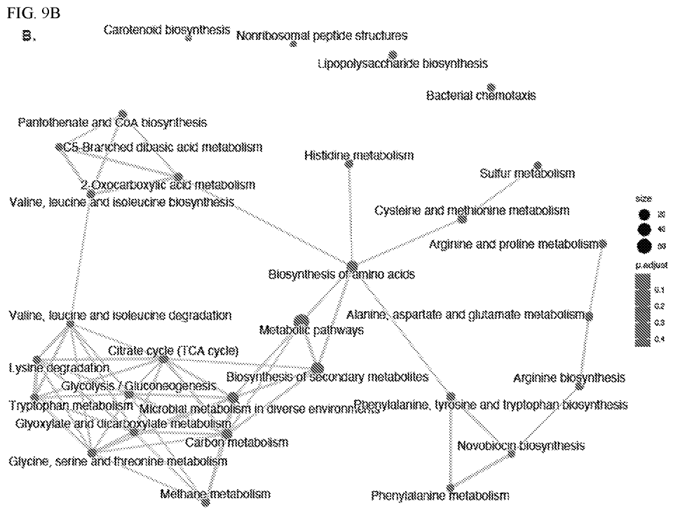

Based on COG annotation, a large proportion of candidate genes functioned in cell envelope biogenesis, motility, amino acid transport and signal transduction (FIG. 3). Enrichment analysis of candidate genes corroborated the COG functional annotation; pathways associated with amino acid and secondary metabolite biosynthesis and bacterial chemotaxis were over-represented in this category (FIG. 9A-B). Of note, a large proportion of the candidate genes have no assigned function, and 20% were annotated as hypothetical or encoding a domain of unknown function.

### Example III

N. musculi Genes Essential for Long Term Colonization are Conserved in Human-Adapted Neisseria

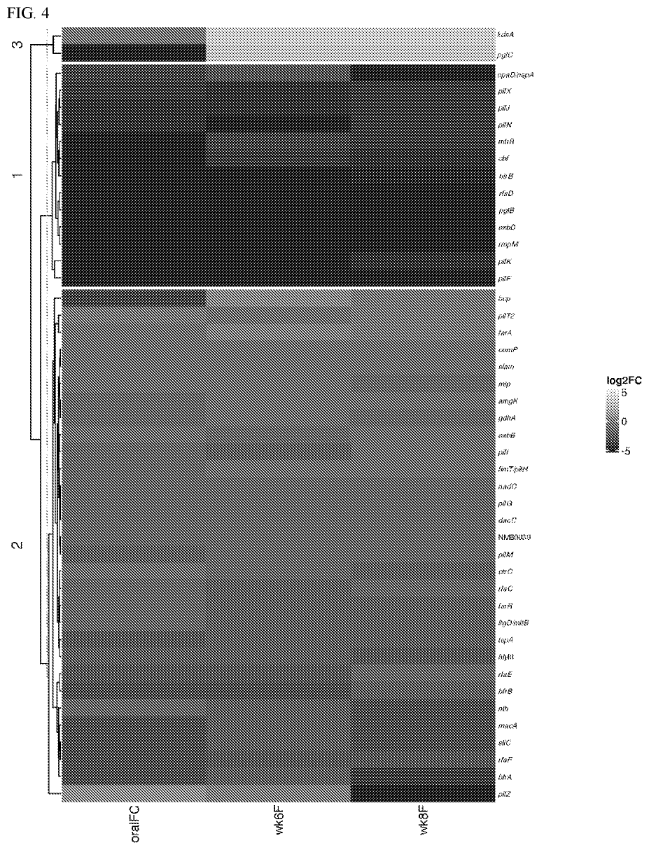

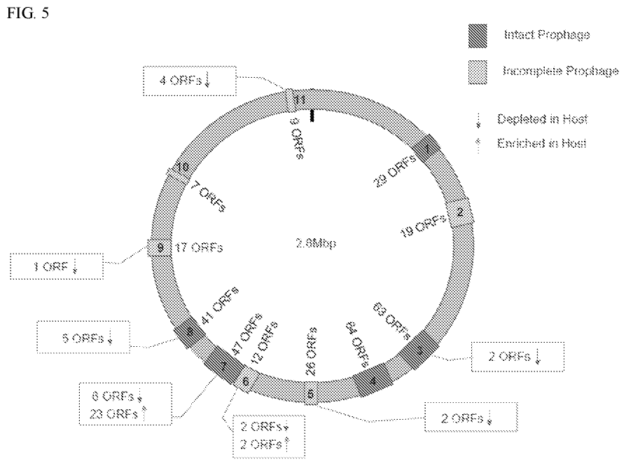

A large collection of genes, termed virulence genes, has been shown to influence the interactions of pathogens Neisseria gonorrhoeae and Neisseria meningitidis with human tissues and cells (Humbert, M. V., et al., Structure of the Neisseria Adhesin Complex Protein (ACP) and its role as a novel lysozyme inhibitor. PLoS Pathog, 2017. 13(6): p. e1006448; Eriksson, J., et al., Characterization of motility and piliation in pathogenic Neisseria. BMC Microbiol, 2015. 15: p. 92; Exley, R. M., et al., Identification of meningococcal genes necessary for colonization of human upper airway tissue. Infect Immun, 2009. 77(1): p. 45-51; Winther-Larsen, H. C., et al., Neisseria gonorrhoeae PilV, a type IV pilus-associated protein essential to human epithelial cell adherence. Proc Natl Acad Sci USA, 2001. 98(26): p. 15276-81; Echenique-Rivera, H., et al., Transcriptome analysis of Neisseria meningitidis in human whole blood and mutagenesis studies identify virulence factors involved in blood survival. PLoS Pathog, 2011. 7(5): p. e1002027; Maiden, M. C. and O. B. Harrison, Population and Functional Genomics of Neisseria Revealed with Gene-by-Gene Approaches. J Clin Microbiol, 2016. 54(8): p. 1949-55; Hey, A., et al., Transcriptional profiling of Neisseria meningitidis interacting with human epithelial cells in a long-term in vitro colonization model. Infect Immun, 2013. 81(11): p. 4149-59; Quillin, S. J. and H. S. Seifert, Neisseria gonorrhoeae host adaptation and pathogenesis. Nat Rev Microbiol, 2018. 16(4): p. 226-240). Many of these were subsequently discovered in commensal Neisseria (Marri, P. R., et al., Genome sequencing reveals widespread virulence gene exchange among human Neisseria species. PLoS One, 2010. 5(7): p. e11835; Snyder, L. A. and N. J. Saunders, The majority of genes in the pathogenic Neisseria species are present in non-pathogenic Neisseria lactamica, including those designated as ‘virulence genes’. BMC Genomics, 2006. 7: p. 128). Curation of 186 virulence genes and 50 genes with significant changes in expression during in vitro infection identified 166 Nmus homologs; 45 of these homologs were identified by the Tn5 screen as essential for long term mouse colonization (FIG. 4). They function in LOS synthesis (rfaCDEF, kdsA), iron acquisition and detoxification (bfrAB, hemolysin III, bcp), efflux (macA,farA), transcriptional regulation (farR), and cell wall recycling, synthesis and repair (amgK, ltgD/mltB, dacC), among others (Knilans, K. J., et al., Neisseria gonorrhoeae Lytic Transglycosylases LtgA and LtgD Reduce Host Innate Immune Signaling through TLR2 and NOD2. ACS Infect Dis, 2017. 3(9): p. 624-633; Schaub, R. E. and J. P. Dillard, The Pathogenic Neisseria Use a Streamlined Set of Peptidoglycan Degradation Proteins for Peptidoglycan Remodeling, Recycling, and Toxic Fragment Release. Front Microbiol, 2019. 10: p. 73; Lee, E. H., et al., FarR regulates the farAB-encoded efflux pump of Neisseria gonorrhoeae via an MtrR regulatory mechanism. J Bacteriol, 2003. 185(24): p. 7145-52; McNeil, G., M. Virji, and E. R. Moxon, Interactions of Neisseria meningitidis with human monocytes. Microb Pathog, 1994. 16(2): p. 153-63; Moore, J., et al., Recognition of saccharides by the OpcA, OpaD, and OpaB outer membrane proteins from Neisseria meningitidis. J Biol Chem, 2005. 280(36): p. 31489-97; Virji, M., et al., Meningococcal Opa and Opc proteins: their role in colonization and invasion of human epithelial and endothelial cells. Mol Microbiol, 1993. 10(3): p. 499-510; Ma, M., et al., A mouse model for Neisseria colonization. Infect Immun, 2018; John, C. M., et al., Lipooligosaccharide Structures of Invasive and Carrier Isolates of Neisseria meningitidis Are Correlated with Pathogenicity and Carriage. J Biol Chem, 2016. 291(7): p. 3224-38; Gisin, J., et al., A cell wall recycling shortcut that bypasses peptidoglycan de novo biosynthesis. Nat Chem Biol, 2013. 9(8): p. 491-3; Chen, C. Y. and S. A. Morse, Neisseria gonorrhoeae bacterioferritin: structural heterogeneity, involvement in iron storage and protection against oxidative stress. Microbiology, 1999. 145 (Pt 10): p. 2967-75; Seib, K. L., et al., Defenses against oxidative stress in Neisseria gonorrhoeae: a system tailored for a challenging environment. Microbiol Mol Biol Rev, 2006. 70(2): p. 344-61). At least one, opaD nspA, encodes an immunomodulatory protein (Shaik, Y. B., et al., Expression of the iron-activated nspA and secY genes in Neisseria meningitidis group B by Fur-dependent and -independent mechanisms. J Bacteriol, 2007. 189(2): p. 663-9). Open reading frames with high similarity to surface associated proteins (mip, slam1) were also required for host persistence (Leuzzi, R., et al., Ng-MIP, a surface-exposed lipoprotein of Neisseria gonorrhoeae, has a peptidyl-prolyl cis/trans isomerase (PPIase) activity and is involved in persistence in macrophages. Mol Microbiol, 2005. 58(3): p. 669-81; Grossman, A. S., et al., A Widespread Bacterial Secretion System with Diverse Substrates. mBio, 2021. 12(4): p. e0195621; Hooda, Y., et al., Slam is an outer membrane protein that is required for the surface display of lipidated virulence factors in Neisseria. Nat Microbiol, 2016. 1: p. 16009).

The screen also identified many Type IV pilus (Tfp) genes. They encode the pilin assembly ATPase (pilF), minor pilins (pilH,I,J,K and comP), the inner membrane Tfp pore complex (pilM,N,G) and pilin glycosylation enzymes (pglB,pglC) (Freitag, N. E., H. S. Seifert, and M. Koomey, Characterization of the pilF-pilD pilus-assembly locus of Neisseria gonorrhoeae. Mol Microbiol, 1995. 16(3): p. 575-86; Lauer, P., N. H. Albertson, and M. Koomey, Conservation of genes encoding components of a type IV pilus assembly/two-step protein export pathway in Neisseria gonorrhoeae. Mol Microbiol, 1993. 8(2): p. 357-68; Watson, A. A., R. A. Alm, and J. S. Mattick, Identification of a gene, pilF, required for type 4 fimbrial biogenesis and twitching motility in Pseudomonas aeruginosa. Gene, 1996. 180(1-2): p. 49-56; Carbonnelle, E., et al., A systematic genetic analysis in Neisseria meningitidis defines the Pil proteins required for assembly, functionality, stabilization and export of type IV pili. Mol Microbiol, 2006. 61(6): p. 1510-22; Winther-Larsen, H. C., et al., A conserved set of pilin-like molecules controls type IV pilus dynamics and organelle-associated functions in Neisseria gonorrhoeae. Mol Microbiol, 2005. 56(4): p. 903-17; Berry, J. L., et al., Functional analysis of the interdependence between DNA uptake sequence and its cognate ComP receptor during natural transformation in Neisseria species. PLoS Genet, 2013. 9(12): p. e1004014; Marceau, M. and X. Nassif, Role of glycosylation at Ser63 in production of soluble pilin in pathogenic Neisseria. J Bacteriol, 1999. 181(2): p. 656-61; Power, P. M., et al., Genetic characterization of pilin glycosylation in Neisseria meningitidis. Microbiology, 2000. 146 (Pt 4): p. 967-79; Goosens, V. J., et al., Reconstitution of a minimal machinery capable of assembling periplasmic type IV pili. Proc Natl Acad Sci USA, 2017. 114(25): p. E4978-E4986; Craig, L., K. T. Forest, and B. Maier, Type IV pili: dynamics, biophysics and functional consequences. Nat Rev Microbiol, 2019. 17(7): p. 429-440). pilZ, whose role in Tfp biogenesis/function has yet to be fully characterized, was significantly underrepresented only in the week 8 mouse-passaged gut sample (Brown, D. R., et al., Systematic functional analysis reveals that a set of seven genes is involved in fine-tuning of the multiple functions mediated by type IV pili in Neisseria meningitidis. Infect Immun, 2010. 78(7): p. 3053-63). Proper elongation of the Tfp fiber requires peptidoglycan remodeling. In this context, it is interesting to note that dacC, encoding a serine type D-ala-D-ala carboxypeptidase, was identified in the screen (Obergfell, K. P., et al., The low-molecular-mass, penicillin-binding proteins DacB and DacC combine to modify peptidoglycan cross-linking and allow stable Type IV pilus expression in Neisseria gonorrhoeae. Mol Microbiol, 2018).

### Example IV

### In Vivo Screen Identifies Novel Neisseria Host Interaction Factors

A closer examination of candidate genes with only a predicted function reveals a high degree of amino acid similarity between their protein products and those of genes in human-adapted Neisseria spp. (Table 2). These genes cover a broad range of functional categories, including cell envelope synthesis and modification, transcriptional regulation, signal transduction and transport. Of interest are cstA (H7A79_RS05505) and gluP (H7A79_RS03010), both of which are encoded by human- and animal-adapted Neisseria and implicated in colonization and virulence in other animal-adapted bacteria that cause zoonosis. cstA, encodes the peptide transporter carbon starvation protein A with 54% identity to Campylobacter jejunii CstA. In this pathogen, the transporter is required for utilization of di- and tri-peptides as nitrogen sources under energy limiting conditions, implicating amino acid utilization as a requirement for host colonization (Rasmussen, J. J., et al., Campylobacter jejuni carbon starvation protein A (CstA) is involved in peptide utilization, motility and agglutination, and has a role in stimulation of dendritic cells. J Med Microbiol, 2013. 62(Pt 8): p. 1135-1143). gluP encodes a glucose/galactose MFS family transporter with 54.5% identity, over 400 amino acids, to Brucella abortus GluP. While gluP has been identified as upregulated in Nine in vitro infection studies, its exact contribution to persistent carriage remains unknown in this context (Schoen, C., et al., Metabolism and virulence in Neisseria meningitidis. Front Cell Infect Microbiol, 2014. 4: p. 114; Muir, A., et al., Construction of a complete set of Neisseria meningitidis mutants and its use for the phenotypic profiling of this human pathogen. Nat Commun, 2020. 11(1): p. 5541). B. abortus gluP mutants have a reduced ability to survive in alternatively activated macrophages and to persist in a chronic mouse infection model in response to PPARγ-dependent modulation of intracellular glucose levels (Xavier, M. N., et al., PPARgamma-mediated increase in glucose availability sustains chronic Brucella abortus infection in alternatively activated macrophages. Cell Host Microbe, 2013. 14(2): p. 159-70). These observations indicate that Nmus gluP and cstA may function in a similar manner to allow long term colonization.

While the vast majority of host essential genes were underrepresented in mouse-passaged samples compared to the inoculum, a very small number were enriched in host outputs. The latter were almost exclusively annotated as components of toxin-antitoxins systems, phage-associated, or within putative prophage regions. Analysis of the Nmus genome using the PHASTER annotation server identified 11 different phage-associated loci, 5 of which were considered complete prophage genomes (FIG. 5, Table 3). Forty-seven genes in the phage-associated regions were classified as significant after mouse passage and 6 others were in the housekeeping candidate pool. Of the 47 ORFs, 22 were depleted in mouse-passaged libraries compared to the inoculum while the others were enriched, suggesting Tn5 disruption of these ORFs improved the ability of Nmus to colonize the mouse. Enriched genes were located in two adjacent clusters. These ORFs were annotated as hypotheticals, phage structural and regulatory components as well as two toxin-antitoxin systems.

Twenty-nine of these open reading frames were found in a single, intact prophage region from nucleotides 1669269-1713960 (region 7, Table 3). The gene organization of this region appeared to be unique to animal Neisseria; a blast comparison revealed the region, comprised of 47 ORFs, to have 87% identity to a similar region in N. animalis (45% query coverage at the nucleotide level). The vast majority of significant genes in this region are enriched; 79% (23/29) of candidate genes in this prophage region had a fold change >1.0 in at least 1 time point and sample site. Notably, prophage region 7 ORFs depleted from mouse-passaged samples tended to be associated with nucleotide modification (when function could be assigned). The only depleted genes in this prophage were H7A79_RS08530, encoding a DNA-methyltransferase; H7A79_RS08575, encoding an HNH endonuclease; H7A79_RS08645, encoding an exonuclease; H7A79_RS08695, encoding putative dCTP deaminase; H7A79_RS08650, encoding a lipoprotein of unknown function; and H7A79_RS08690, encoding a hypothetical protein. These genes may be required for maintenance of host immunity or temperate lifecycles, as their loss appeared to negatively impact Nmus passage through the mouse.

The remaining 16 phage-associated host essential candidates were located within regions 3, 5, 6, 9 and 11. Of interest in these clusters are homologs encoding the cold shock protein (cspA) and ssrA binding protein (smpB), which may be involved in post-transcriptional regulation of stress responses, in region 11; and a GpeE family phage tail chaperone protein, in region 3. Common to all phage regions containing host essential genes was the prevalence of hypothetical ORFs that were likely derived from a bacterial host. Nine of 21 host essential genes in these regions had no assigned function and 7 lacked similarity to a phage gene product. This underscores the potential role of phage-mediated horizontal gene transfer in transporting host interaction factors between bacteria occupying a similar niche.

### Example V

The Capsular Polysaccharide Promotes N. musculi Colonization of the Oral Cavity

Four genes annotated as components of a capsular polysaccharide (cps) were underrepresented in the mouse-passaged samples compared to the inoculum. Although the capsule is widely considered a virulence factor, cps loci were recently reported in commensal Neisseria including Nmus (Clemence, M. E. A., M. C. J. Maiden, and O. B. Harrison, Characterization of capsule genes in non-pathogenic Neisseria species. Microb Genom, 2018. 4(9); Ma, M., et al., A Natural Mouse Model for Neisseria Colonization. Infect Immun, 2018. 86(5)). The presence of Nmus cps genes in the Tn5 screen provides an opportunity to test the importance of this extracellular component to commensal colonization.

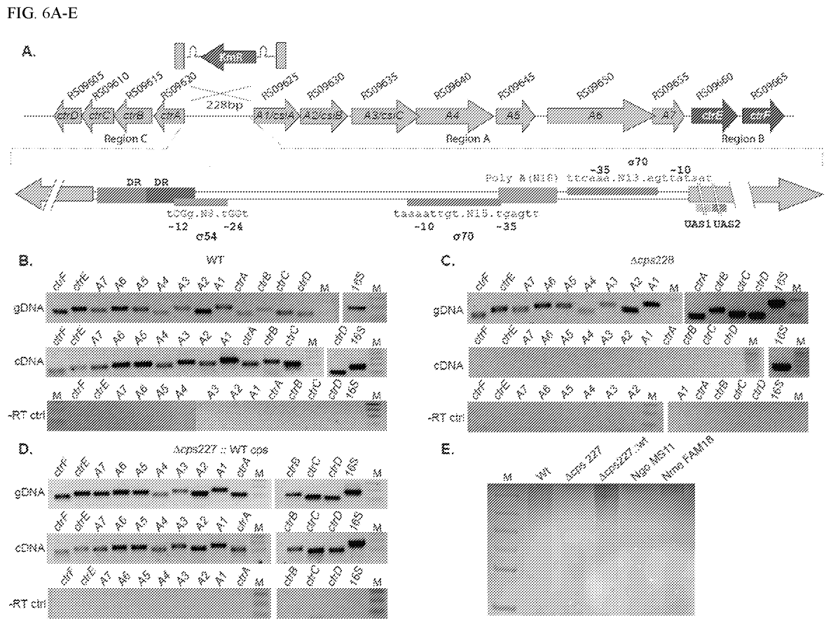

Nmus cps genes (FIG. 6) are analogous to those in N. meningitidis (Nine), with assigned functions in capsule synthesis (region A), transport (region B), and translocation (region C) (Harrison, O. B., et al., Description and nomenclature of Neisseria meningitidis capsule locus. Emerg Infect Dis, 2013. 19(4): p. 566-73). The gene order of region C and B is identical to the order found in all other Neisseria species. The genes and organization of region A is more divergent (FIG. 6A), although it carries genes with annotated functions similar to Nine serogroup I and clusters with other animal Neisseria region A genes (Clemence et al., supra).

The 4 Nmus cps genes underrepresented in mouse-passaged samples are situated in region C (H7A79_RS9610, encoding an ABC transporter permease homolog of CtrC) and region A (H7A79_RS9625, encoding a CsiA homolog annotated as a UDP-N-acetylglucosamine epimerase (A1); H7A79_RS09645, encoding a putative rhamnosyl transferase (A5), and H7A79_RS09655, encoding a putative glycosyltransferase (A7)) (Table 2).

In region C and region A, start and stop codons of adjacent ORFs are often close together and in some cases overlap. The regulatory sequences for region C and A are likely located in a 228 bp segment of DNA between the two gene clusters; this intergenic region contains putative Sigma54 and Sigma70 consensus sequences, an Npa upstream activator sequence, as well as a 17-bp direct repeat and a 20-bp poly-Thymine tract, either or both of which may also serve as regulatory sequences (FIG. 6A).

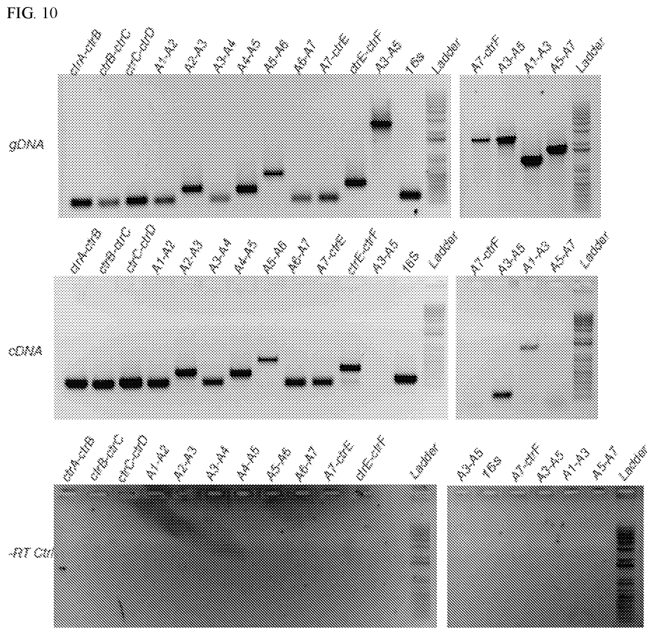

To determine whether the 228 bp intergenic region regulates expression of the capsule clusters, RT-PCR was performed on RNA isolated from log phase Nmus AP2365, using different combinations of primers spanning the junctions of consecutive ORFs. The genes in region A (csiA A1-A7) and region B (ctrE, ctrF) are transcribed as a single mRNA. Genes in region C (ctrA,B,C,D) are divergently transcribed, also as a single mRNA. This indicates that transcription of the two operons is likely to be controlled from the 228 bp csiA A1-ctrA intergenic region (FIG. 10).

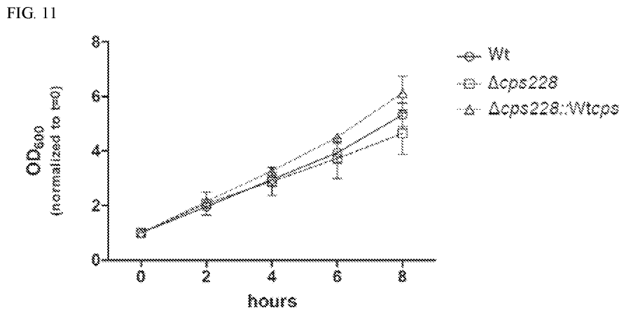

To validate the essentiality of the capsule for long term mouse colonization, the 228 bp intergenic region in Wt Nmus AP2365 was replaced with a kanamycin cassette, generating Δcps228. Deletion of this putative regulatory element resulted in loss of capsule production in the mutant (FIG. 6B-D). RT-PCR experiments using RNA from Wt Nmus, Δcps228 and complemented strains demonstrated that loss of the csiA A1-ctrA intergenic region ablated transcription of all genes in regions A, B and C. Compared to Wt, complement and Nine FAM18 controls, extracts from Δcps228 did not react with the capsule stain Alcian Blue (FIG. 6E). Loss of capsule did not affect the in vitro growth rate of the mutant in comparison to Wt and complemented controls (FIG. 11).

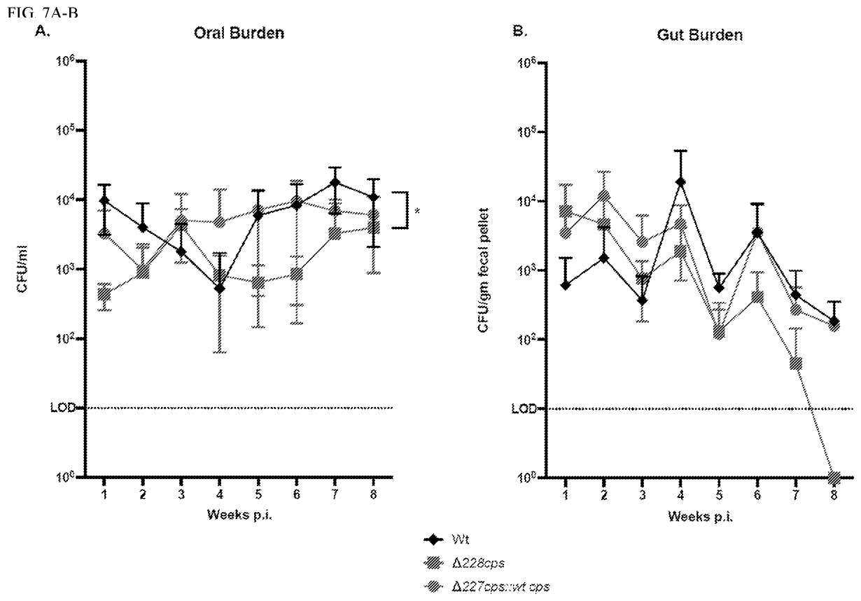

Wt Nmus, Δcps228, and complemented strains were inoculated into A/J mice to validate mutant phenotype independent of potential mouse strain background factors. Nmus burdens from oral and fecal samples were assessed weekly for 8 weeks (FIG. 7). No difference in absolute colonization frequency was observed between Wt, mutant and complemented control groups. However, starting 4 weeks post-inoculation, oral cavity burdens of Δcps228 began to decline in comparison to the Wt and complemented strains. This downward trend continued through the remaining 4 weeks of the study, and a significant difference in weekly Δcps228 CFUs was observed in comparison to the controls. Together, these results indicate that while unencapsulated Nmus may be able to colonize the oral cavity of mice, they are at a significant disadvantage in long term colonization.

TABLE 1
Primers, Synthetic sequences, and Strains utilized in this study

| | | SEQ ID | |
|---|---|---|---|
| Primer | Use | NO.: | Sequence |
| Ind_Ad_T | Library | 27 | ACACTCTTTCCCTACACGACGCTCTTCCGATC*T |
| | preparation | | |
| Ind_Ad_B | Library | 28 | GATCGGAAGAGCGGTTCAGCAGGAATGCCGAGACCGATCTC |
| | preparation | | |
| Tn-FO | Tn5 Insertion | 29 | TCGTCGGCAGCGTCAGATGTGTATAAGAGACAGCGGGGATCCT |
| | site enrichment | | CTAGAGTCGACCTGC |
| Adapt-RO | PCR | 30 | GTCTCGTGGGCTCGGAGATGTGTATAAGAGACAGACACTCTTT |
| | | | CCCTACACGACGCTCTTCCGATC |
| MR493 | Detect 16S | 31 | ATCCTGGCTCAGATTGAACG |
| MR494 | transcript | 32 | CCGCTTTCCTTCTCAAAGTG |
| IM017 | Detect ctrA | 33 | AGTATCCGTATGCCGCTCAC |
| IM018 | transcript | 34 | AGCTGCACCGAAATATCCTG |
| IM086 | Detect ctrB | 35 | TTAAAGAAGCGGCAGGAGAG |
| IM087 | transcript | 36 | ACTTGGTCAAGCTGGGTTTG |
| IM088 | Detect ctrC | 37 | ATGCGGTAGGAAAACTGTGG |
| IM089 | transcript | 38 | ACCCACAAAGCGTATTCCTG |
| IM090 | Detect ctrD | 39 | CCGACTAAAGGCGAGATCAG |
| IM091 | transcript | 40 | AAAGCGCAAATTATCCATGC |
| IM019 | Detect A1 | 41 | CAGATGGACGTTGTGTTTGC |
| IM020 | transcript | 42 | AGGGAAAATTTCGGAGAAGG |
| IM094 | Detect A2 | 43 | ATTGATCTCACTGGCCAACC |
| IM095 | transcript | 44 | GGTCGGGTGTTTTATTCACG |
| IM096 | Detect A3 | 45 | ACAGCGTAACCAGACCTTGG |
| IM097 | transcript | 46 | AGCCGGGTCTTCTTTATTCC |
| IM098 | Detect A4 | 47 | TCCTGCCAATCGGATTTTAG |
| IM099 | transcript | 48 | TTTAATCAGGCTGGGTACGG |
| IM100 | Detect A5 | 49 | TGGGGCAGTAGCTTTCTTTC |
| IM101 | transcript | 50 | AGGGCAGCAATACCTTTTCC |
| IM102 | Detect A6 | 51 | CGACCATAAATGGGGTGTTC |
| IM103 | transcript | 52 | ATGAACAGCGGCAGGTAATC |
| IM104 | Detect A7 | 53 | TTCGACCGCTTTATCGATTC |
| IM105 | transcript | 54 | ACGGGAAACATCGCTACATC |
| IM021 | Detect ctrE | 55 | CATAAAAAGGCAGGCCGTAG |
| IM022 | transcript | 56 | GACCAAACCGTAACCGAATG |
| IM023 | Detect ctrF | 57 | AGCTGCCTTGAAAGGTGATG |
| IM024 | transcript | 58 | GTTACGCTCAACAGCACCAG |
| IM110 | Detect ctrA- | 59 | CGCCGATTGTCAGTAGTGTG |
| IM111 | ctrB transcript | 60 | TAGCAGCTGTTGCCTGTTTC |
| IM112 | Detect ctrB- | 61 | ATCGGTTTGATGCTTTACGG |
| IM113 | ctrC transcript | 62 | TTACCCGCCCTTGAATAACC |
| IM114 | Detect ctrC- | 63 | CAAGTTCAGCAAAGGGGTTG |
| IM115 | ctrD transcript | 64 | AGAATGCCGATTTTCTCACC |
| IM116 | Detect A1-A2 | 65 | TACGCCGATAACTTTTACGC |
| IM117 | transcript | 66 | TGCCATTTAACGAGGAAAGC |
| IM118 | Detect A2-A3 | 67 | TTCTCAAGCTGTTCATCATCG |
| IM119 | transcript | 68 | TGTAGATTCCGTGGTGGTTG |
| IM120 | Detect A3-A4 | 69 | CTTTAGGGAGGCAATACATGC |
| IM121 | transcript | 70 | TCGGCCAAACAATGGATTAC |
| IM122 | Detect A4-A5 | 71 | CTGAAAACAGATGGGCAATG |
| IM123 | transcript | 72 | AACGGTCAGCGGACAAAG |
| IM124 | Detect A5-A6 | 73 | GTTAAGGCATGGGCAGTTTG |
| IM125 | transcript | 74 | ACCGCCAATGGAACATTATC |
| IM126 | Detect A6-A7 | 75 | GGAGGGAAAACCAACTGCTG |
| IM127 | transcript | 76 | TCTTAGAAGCAGAGATCAACAAGG |
| IM128 | Detect A7-ctrE | 77 | AAGCCCGAAACTTGAGAAAAG |
| IM129 | transcript | 78 | GATTTGGAAATGGCCAAGG |
| IM130 | Detect ctrE-ctrF | 79 | TCCGGGAATAAAGGTGTTTG |
| IM131 | transcript | 80 | AAACCGCCGTCAATATTCTG |
| IM132 | Detect Al-A3 | 81 | GATTCCGGCGGTATTCAAG |
| IM133 | transcript | 82 | TCCTGTTGGAAATAACGTTTCA |
| IM134 | Detect A3-A5 | 83 | TGACGGAAATCGTATTGCTTT |
| IM135 | transcript | 84 | ACTCATTTCGGCCAAACAAT |
| IM136 | Detect A3-A5 | 85 | CTGAAAACAGATGGGCAATG |
| IM137 | (1) transcript | 86 | GCACAAAAATTCACCGAAGC |
| IM138 | Detect A5-A7 | 87 | CGGCGGGTATGCAGAATC |
| IM139 | transcript | 88 | ACAAACCGGCACAGCTTACT |
| IM140 | Detect A7-ctrF | 89 | AGCCATTGCGAGAAGGTTTT |
| IM141 | transcript | 90 | AAGCGGCAGCTTTATTTGAA |
| IM059 | Detect | 91 | TGCCGAATACAATCAGCAGT |
| IM060 | Δcps227::Kan | 92 | TCCACATCAACCACAGCAAC |
| | mutant | | |
| IM132 | Deletion of the | 93 | TTTGATAGCCTCCGGCCGGGTGCCGAATACAATCAGCAGTT |
| | 228bp IGR | | TTTTAGCACGTGCGGGGGGAGGGGATACCGCTATGGGATT |
| | | | AAAAGCAAATGAATCGTTGAATGTTGTCAATGTCAGAGCAC |
| | | | TATTATCGGCGTTATTGTTTTTTTGCACGGCACAGCCTTTCG |
| | | | TATTACACGGGTAGAACTTTGTCATTTAAGGGATGCAGTTTA |
| | | | TGCATCCCTTAACTCGAGGGCTTGACACTTTATGCTTCCGGCTC |
| | | | GTATAATGTGTGGATAGTGGGAGGAAAGCATGATTGAACAAGA |
| | | | TGGATTGCACGCAGGTTCTCCGGCCGCTTGGGTGGAGAGGCTA |
| | | | TTCGGCTATGACTGGGCACAACAGACAATCGGCTGCTCTGATG |
| | | | CCGCCGTGTTCCGGCTGTCAGCGCAGGGGCGCCCGGTTCTTTTT |
| | | | GTCAAGACCGACCTGTCCGGTGCCCTGAATGAACTGCAGGACG |
| | | | AGGCAGCGCGGCTATCGTGGCTGGCCACGACGGGCGTTCCTTG |
| | | | CGCAGCTGTGCTCGACGTTGTCACTGAAGCGGGAAGGGACTGG |
| | | | CTGCTATTGGGCGAAGTGCCGGGGCAGGATCTCCTGTCATCCC |
| | | | ACCTTGCTCCTGCCGAGAAAGTATCCATCATGGCTGATGCAAT |
| | | | GCGGCGGCTGCATACGCTTGATCCGGCTACCTGCCCATTCGACC |
| | | | ACCAAGCGAAACATCGCATCGAGCGAGCACGTACTCGGATGGA |
| | | | AGCCGGTCTTGTCGATCAGGATGATCTGGACGAAGAGCATCAG |
| | | | GGGCTCGCGCCAGCCGAACTGTTCGCCAGGCTCAAGGCGCGCA |
| | | | TGCCCGACGGCGAGGATCTCGTCGTGACCCATGGCGATGCCTG |
| | | | CTTGCCGAATATCATGGTGGAAAATGGCCGCTTTTCTGGATTCA |
| | | | TCGACTGTGGCCGGCTGGGTGTGGCGGACCGCTATCAGGACAT |
| | | | AGCGTTGGCTACCCGTGATATTGCTGAAGAGCTTGGCGGCGAA |
| | | | TGGGCTGACCGCTTCCTCGTGCTTTACGGTATCGCCGCTCCCGA |
| | | | TTCGCAGCGCATCGCCTTCTATCGCCTTCTTGACGAGTTCTTCT |
| | | | GAGCCGTCTGAAGTTTAAACATCGATAAAATGCCGTCCGAAGC |
| | | | TTGAGTTCAGACGGCATTTTATGACGCGTTTCCAGTTGCCGG |
| | | | CCGGATGCATTCTATTGCTGCTTTTAGGGTGTGGTGCTTTG |
| | | | CCTACTTCAGGGCCGAATCAAACCAAGGTGATAAGTTTGCA |
| | | | AAATCAAAGCCGCCAGGCGGCAGTGCCTGATGTTGCTGTG |
| | | | GTTGATGTGGACGAGCGGGTAATTTCCACACTGTAT |
| IM133 | Capsule | 94 | ACGCCTTCACGAAACAGATT |
| IM134 | complementation | 95 | AATTATTCTGCGCCACACG |
| Strain | Species | | Notes |
| AP2365 | N. musculi | | Rif resistant derivative of AP2031 type strain |
| AP2365 | N. musculi | | |
| Δcps228 | | | |
| AP2365Δcp | N. musculi | | |
| s228::wt | | | |
| cps | | | |
| MS11 | N. gonorrhoeae | | |
| FAM18 | N. meningitidis | | |

TABLE 2
A selection of candidates identified as essential for host passage.

| | GenBank | | | Avg Fold | Pathway | |
|---|---|---|---|---|---|---|
| RefSeq Locus | Locus | Name | Annotation | Change | Association | Neisseria homologs⁺ (% Identity) |
| H7A79_RS9625 | H7A79_1831 | A1/csiA | UDP-N- | −2.2 | capsule | Nzo(78.6), Nme*(72) |
| | | (see | acetylglucosamine | | synthesis | |
| | | FIG. 6) | epimerase | | | |
| H7A79_RS09645 | H7A79_1835 | A5 | hypothetical protein, | −3.0 | capsule | Nci(64.8) |
| | | (see | putuative rhamnosyl | | synthesis | |
| | | FIG. 6) | transferase | | | |
| H7A79_RS09655 | H7A79_1837 | A7 | Glycosyltransferase | −4.4 | capsule | Nme(84), Nci(79) |
| | | (see | | | synthesis | |
| | | FIG. 6) | | | | |
| H7A79_RS03350 | H7A79_0628 | mreD | Rod shape-determining | −1.6 | Type II TA | Nel(85.3), Nani(97.7) |
| | | | protein | | system related | |
| | | | | | factors | |
| H7A79_RS03345 | H7A79_0627 | mreC | Rod shape-determining | −2.7 | Chromosome | Nel(62.0), Nsf(74.4), Nfv(55.1) |
| | | | protein | | partitioning | |
| | | | | | proteins | |
| H7A79_RS07140 | H7A79_1361 | tig | trigger factor | −6.3 | protein | Nzo(87.4), Nla(79.4), |
| | | | | | processing | Nsc(79.6), Nmj(79.4), |
| | | | | | | Ncz(79.1), Nme(79.6), Ngo(78.4) |
| H7A79_RS10380 | H7A79_1976 | oxyR | Hydrogen peroxide- | −4.2 | Transcription | Nme(88.5), Ngo(88.2), |
| | | | inducible genes | | factors | Nel(84.6)Nsi(88.5), |
| | | | activator | | | Nmj(88.9), Nsf(89.8), |
| | | | | | | Nfv(89.5), Nla(88.8), |
| | | | | | | Nani(88.7), Nci(86.7) |
| H7A79_RS03110 | H7A79_0582 | | tetR family | −3.1 | Transcriptional | Nzo(84.1), Nsf(79.4), |
| | | | transcriptional | | regulation | Ngo(78.6), Nfv(78), |
| | | | regulator | | | Nla(77.7), Nme(76.8), |
| | | | | | | Nsi(79.4) |
| H7A79_RS07670 | H7A79_1461 | bolA | bolA family | −3.8 | Transcriptional | Nzo(77), Nmj(69.7), Nsi(69.7), |
| | | | transcriptional | | regulation | Nfv(67.0), Nsf(65.9), Nel(60.2), |
| | | | regulator | | | Nla(56.2), Nme(59.5), Ngo(58.3) |
| H7A79_RS14490 | H7A79_2775 | | MarR family | −7.8 | Transcriptional | Nmj(81.5), Nsi(81.5), Nfv(78.5), |
| | | | transcriptional | | regulation | Nel(53.1) Nsc(81.5) |
| | | | regulator | | | |
| H7A79_RS01540 | H7A79_0286 | matE | Multidrug transporter | −3.2 | Transport | Nsi(54), Nmj(53.3), Nsf(43.8), |
| | | | | | | Nci(47.4), Nani(61) |
| H7A79_RS03010 | H7A79_0563 | gluP | Glucose/galactose MFS | −4.7 | Transport | Nme(74.9), Ngo(74.4), Nsf(74.4) |
| | | | transporter | | | |
| H7A79_RS04300 | H7A79_0825 | mscL | Large conductance | −6.7 | Transport | Nel(82.9), Nsf(86.7), Nci(81.4), |
| | | | mechanosensitive | | | Ncz(80.1), Nmj(84.5), Nfv(84.4) |
| | | | channel protein | | | |
| H7A79_RS06400 | H7A79_1225 | | Entericidin A/B family | −4.3 | Transport | Nmj(65), Nani(88.6), Nci(81.8) |
| | | | lipoprotein | | | |
| H7A79_RS12155 | H7A79_2323 | | oligopeptide ABC | −2.9 | Transport | Nani(78.3), Nwe(78.2), |
| | | | transporter substrate- | | | Nzo(78.0), Nzl(76.8) |
| | | | binding protein OppA | | | |
| H7A79_RS14545 | H7A79_2785 | | putative membrane | −2.3 | Transport | Nzo(85.7), Nsg(81.6), Nwd(88.3), |
| | | | protein/divalent metal | | | Ncz(61.4), Nsf(61.1) |
| | | | cation transporter | | | |
| H7A79_RS05505 | H7A79_1050 | cstA | carbon starvation | −4.8 | Transport | Nzo(92.2), Nsf(85.3), |
| | | | protein A | | | Nel(85.7), Nbc(85.4), |
| | | | | | | Nla(85.8), Nmj(85.2), |
| | | | | | | Ncz(85.4), Nme(85.7) |
| H7A79_RS00105 | H7A79_0023 | cheY | Response regulator | −3.2 | Two component | Nci(74.4), Nbc(74.1) |
| | | | | | systems, motility | |
| H7A79_RS00110 | H7A79_0024 | cheX | Chemotaxis protein | −1.7 | Two component | Nani(83.1), Nci(72.5), |
| | | | CheX | | systems, motility | Nbc(66) |
| H7A79_RS14225 | H7A79_2721 | | response regulator | −8.1 | Signal | Nzo(82.1), Nsf(75.7), Nfv(76.0), |
| | | | | | transduction | Nmj(73.2), Nsi(73.2), Nme(59.0) |
| H7A79_RS10685 | H7A79_2032 | | cyclic nucleotide-binding | −2.2 | signaling | Nwd(67.2), Nbl(60.1), Nzo(83.2) |
| | | | domain-containing | | | |
| | | | protein | | | |
| H7A79_RS01525 | H7A79_0283 | | vanZ family protein | −2.5 | unknown | Ngo(55.8), Nsi(56.0), Nsf(58.5), |
| | | | | | | Nfv(57.6), Nmj(56), Nani(66.1) |
| H7A79_RS02820 | H7A79_0527 | | yecA family protein | −7.0 | unknown | Nzo(80.6), Nsf(71.5), |
| | | | | | | Nfv(71.5), Nsi(71.6), |
| | | | | | | Nla(66.4), Ngo(65.9), Nme(65.9) |
| H7A79_RS07070 | H7A79_1349 | | conjugal transfer | −2.5 | unknown | Nzo(80.1), Nel(61.8), |
| | | | protein | | | Nsc(61.1), Nsf(58.2), |
| | | | | | | Nfv(58.2), Ncz(57.8), |
| | | | | | | Nla(58.2), Nme(57.9) |
| H7A79_RS12320 | H7A79_2356 | rhuM | Virulence factor | −2.1 oral | unknown | Nme(51), Nfv(44), Nel(45), |
| | | | | only | | Nla(51), Nmj(45) |
| H7A79_RS13690 | H7A79_2619 | | M61 family | −1.6 oral & | unknown | Ngo(65.1), Nme(64.7), Nmj(67), |
| | | | metallopeptidase | wk6 | | Nel(66.2), Nfv(64.8), Nla(64.4), |
| | | | | | | Nsf(64.1), Nani(70.6), Nci(64) |
| H7A79_RS14210 | H7A79_2718 | | roadblock/LC7 domain | −7.4 | unknown | Nzo(96.7), Nmj(91.5), |
| | | | containing protein | | | Nsi(91.5), Nfv(89.8), |
| | | | | | | Nsf(88.1), Nla(78.0), |
| | | | | | | Ngo(76.3), Nme(76.3) |
| H7A79_RS04855 | H7A79_0925 | | Rid family | −3.7 | Translational | Nzo(91.3), Nel(84.9), |
| | | | endoribonuclease | | regulation | Nmj(87.3), Nsc(87.3), |
| | | | | | | Nsf(87.3), Ncz(87.3), |
| | | | | | | Nsi(86.5), Nme(86.5), |
| | | | | | | Ngo(83.3) |

⁺Genes are grouped by putative pathway association identified by KEGG functional annotation. Amino acid sequence comparisons were computed from KEGG Ortholog GFIT database and BLAST. Species abbreviations are shown according to KEGG nomenclature.
Nme, Neisseria meningitidis MC58; Nme*, Neisseria meningitidis serogroup I/93004; Nel, Neisseria elongata sups. glycolitica ATCC29315; Ngo, Neisseria gonorrhoeae FA1090; Nla, Neisseria lactamica 02-06; Nsi, Neisseria mucosa FDAARGOS; Nfv, Neisseria flavescens ATCC 13120; Ncz, Neisseria cinerea NCTC10294; Nci, Neisseria canis NCTC10296; Nbc, Neisseria baciliformis DSM 233383; Nmj, Neisseria mucosa ATCC 19696; Nani, Neisseria animaloris NCTC12227; Nsf, Neisseria subflava ATCC 49275; Nzo, Neisseria zoodegmatis NCTC12230; Nsg, Neisseria shayeganii DSM22244; Nsc, Neisseria sicca NS20201025; Nwd, Neisseria wadsworthii DSM22245; Nwe, Neisseria weaveri NCTC13585.

TABLE 3
Open reading frames within prophage-like sequences identified by in vivo screening of Nmus Tn5 libraries.

| | | | | | | | | |
|---|---|---|---|---|---|---|---|---|
| | | | | | | | Gut | Gut |
| Genbank | RefSeq | | Phage | | | | Wk 6 | Wk 8 |
| ID | ID | Annotation | Annotation | Region | Type | Oral FC | FC | FC |

Depleted ORFs

| | | | | | | | | |
|---|---|---|---|---|---|---|---|---|
| n/a | H7A79_{—} | GpE family phage tail | n/a | 3 | intact | −4.0 | −5.2 | −7.4 |
| | RS05220 | protein | | | | | | |
| H7A79_{—} | H7A79_{—} | hypothetical | n/a | 3 | intact | −4.6 | −3.4 | −4.8 |
| 1027 | RS05395 | protein | | | | | | |
| H7A79_{—} | H7A79_{—} | DoxX family | PHAGE_Prochl_P_TIM68_NC_028955: | 5 | incomplete | −2.7 | −3.5 | −2.2 |
| 1378 | RS07240 | protein | hypothetical protein | | | | | |
| H7A79_{—} | H7A79_{—} | hemerythrin | n/a | 5 | incomplete | −2.4 | ns | ns |
| 1379 | RS07245 | domain- | | | | | | |
| | | containing protein | | | | | | |
| H7A79_{—} | H7A79_{—} | helix-turn-helix | PHAGE_Thermu_P23_77_NC_013197: | 6 | incomplete | −2.9 | −3.2 | −2.7 |
| 1640 | RS08395 | domain- | hypothetical protein | | | | | |
| | | containing protein | | | | | | |
| n/a | H7A79_{—} | tyrosine-type | PHAGE_Entero_mEpX2_NC_019705: | 6 | incomplete | −4.5 | −7.4 | −7.2 |
| | RS08405 | recombinase/integrase | integrase | | | | | |
| H7A79_{—} | H7A79_{—} | site-specific DNA- | PHAGE_Pseudo_H103_{—} | 7 | intact | −2.9 | −3.0 | −2.0 |
| 1628 | RS08530 | methyltransferase | NC_028819: | | | | | |
| | | | adenine-specific DNA | | | | | |
| | | | methyltransferase | | | | | |
| n/a | H7A79_{—} | HNH | PHAGE_Pseudo_PAJU2_NC_011373: | 7 | intact | −2.1 | ns | ns |
| | RS08575 | endonuclease | putative endonuclease | | | | | |
| H7A79_{—} | H7A79_{—} | hypothetical | PHAGE_Mannhe_vB_MhM_1127AP1_{—} | 7 | intact | ns | −2.3 | ns |
| 1651 | RS08645 | protein | NC_047750: exonuclease | | | | | |
| n/a | H7A79_{—} | hypothetical | PHAGE_Salmon_SSU5_NC_018843: | 7 | intact | −1.9 | ns | ns |
| | RS08650 | protein | putative lipoprotein | | | | | |
| H7A79_{—} | H7A79_{—} | hypothetical | n/a | 7 | intact | ns | −3.3 | ns |
| 1659 | RS08690 | protein | | | | | | |
| H7A79_{—} | H7A79_{—} | dCTP | PHAGE_Pseudo_PA7_NC_042060: | 7 | intact | −2.7 | −2.3 | −4.9 |
| 1660 | RS08695 | deaminase | prohead protease | | | | | |
| H7A79_{—} | H7A79_{—} | integrase arm-type | PROPHAGE_Escher_CFT073: | 8 | intact | −3.4 | −3.9 | −4.0 |
| 1762 | RS09245 | DNA-binding protein | putative prophage integrase | | | | | |
| H7A79_{—} | H7A79_{—} | hypothetical | n/a | 8 | intact | −4.0 | ns | ns |
| 1764 | RS09255 | protein | | | | | | |
| H7A79_{—} | H7A79_{—} | hypothetical | n/a | 8 | intact | −1.9 | ns | ns |
| 1766 | RS09265 | protein | | | | | | |
| H7A79_{—} | H7A79_{—} | hypothetical | n/a | 8 | intact | −3.7 | −3.6 | −4.4 |
| 1796 | RS09440 | protein | | | | | | |
| n/a | H7A79_{—} | pseudogene | PROPHAGE_Neisse_MC58: | 8 | intact | −2.3 | −2.6 | −2.7 |
| | RS09445 | | IS1016C2 transposase | | | | | |
| H7A79_{—} | H7A79_{—} | hypothetical | n/a | 9 | incomplete | −2.9 | −8.6 | −8.4 |
| 1994 | RS10470 | protein | | | | | | |
| H7A79_{—} | H7A79_{—} | cold-shock | PHAGE_Lactoc_bIL312_NC_002671: Csp | 11 | incomplete | −6.0 | −4.0 | −5.2 |
| 2748 | RS14355 | protein | | | | | | |
| H7A79_{—} | H7A79_{—} | DUF1653 domain- | PHAGE_Bacill_PM1_NC_020883: | 11 | incomplete | −6.0 | −5.4 | −10.6 |
| 2749 | RS14360 | containing protein | hypothetical protein | | | | | |
| H7A79_{—} | H7A79_{—} | SsrA-binding protein | PHAGE_Escher_500465_1_NC_049342: | 11 | incomplete | −1.9 | −1.6 | −2.4 |
| 2750 | RS14365 | SmpB | HNH endonuclease | | | | | |
| H7A79_{—} | H7A79_{—} | hypothetical | n/a | 11 | incomplete | ns | −5.7 | ns |
| 2752 | RS14375 | protein | | | | | | |

Enriched ORFs

| | | | | | | | | |
|---|---|---|---|---|---|---|---|---|
| H7A79_{—} | H7A79_{—} | hypothetical | n/a | 6 | incomplete | 1.9 | 1.3 | 1.6 |
| 1609 | RS08435 | protein | | | | | | |
| H7A79_{—} | H7A79_{—} | hypothetical | PHAGE_Acinet_WCHABP12_NC_041924: | 6 | incomplete | 1.6 | ns | 1.4 |
| 1610 | RS08440 | protein | hypothetical protein | | | | | |
| H7A79_{—} | H7A79_{—} | DUF1983 domain- | PHAGE_Mannhe_vB_MhS_{—} | 7 | intact | 1.6 | 1.2 | 1.9 |
| 1614 | RS08460 | containing protein | 535AP2_NC_028853: | | | | | |
| | | | host specificity protein J | | | | | |
| H7A79_{—} | H7A79_{—} | phage minor | PHAGE_Entero_lambda_NC_001416: | 7 | intact | 2.0 | 1.2 | 2.0 |
| 1617 | RS08475 | tail protein L | tail component | | | | | |
| H7A79_{—} | H7A79_{—} | phage tail | PHAGE_Entero_mEp235_NC_019708: | 7 | intact | 1.4 | ns | 1.7 |
| 1618 | RS08480 | protein | minor tail protein M | | | | | |
| H7A79_{—} | H7A79_{—} | phage tail tape | PHAGE_Pseudo_MP29_NC_011613: | 7 | intact | 1.7 | 1.2 | 1.6 |
| 1619 | RS08485 | measure family protein | putative tail component protein | | | | | |
| H7A79_{—} | H7A79_{—} | phage head closure | PHAGE_Entero_mEp235_NC_019708: | 7 | intact | 1.4 | ns | 1.3 |
| 1625 | RS08515 | protein | hypothetical protein | | | | | |
| H7A79_{—} | H7A79_{—} | HicA family | PHAGE_Mannhe_vB_MhS_{—} | 7 | intact | 1.6 | 1.0 | 1.4 |
| 1626 | RS08520 | toxin | 535AP2_NC_028853: | | | | | |
| | | | toxin component HicA | | | | | |
| H7A79_{—} | H7A79_{—} | HicB family | PHAGE_Mannhe_vB_MhS_{—} | 7 | intact | 1.7 | 1.1 | 1.5 |
| 1627 | RS08525 | antitoxin | 535AP2_NC_028853: | | | | | |
| | | | antitoxin component HicB | | | | | |
| H7A79_{—} | H7A79_{—} | hypothetical | n/a | 7 | intact | 1.2 | ns | 1.4 |
| 1629 | RS08535 | protein | | | | | | |
| H7A79_{—} | H7A79_{—} | hypothetical | PHAGE_Paraco_Shpa_NC_041868: | 7 | intact | 1.8 | ns | ns |
| 1631 | RS08545 | protein | hypothetical protein | | | | | |
| H7A79_{—} | H7A79_{—} | phage major capsid | PHAGE_Pseudo_D3_NC_002484: | 7 | intact | 1.6 | ns | 1.7 |
| 1632 | RS08550 | protein | major capsid protein | | | | | |
| H7A79_{—} | H7A79_{—} | HK97 family phage | PHAGE_Entero_phiP27_NC_003356: | 7 | intact | 1.5 | 1.2 | 1.8 |
| 1633 | RS08555 | prohead protease | putative prohead protease | | | | | |
| H7A79_{—} | H7A79_{—} | phage portal | PHAGE_Entero_mEp235_NC_019708: | 7 | intact | ns | 1.9 | 2.2 |
| 1634 | RS08560 | protein | portal protein | | | | | |
| H7A79_{—} | H7A79_{—} | terminase | PHAGE_Burkho_Bcep176_NC_007497: | 7 | intact | 1.7 | 1.1 | 1.4 |
| 1635 | RS08565 | large subunit | gp79 | | | | | |
| H7A79_{—} | H7A79_{—} | hypothetical | PHAGE_Vibrio_J2_NC_027393: | 7 | intact | 1.5 | ns | 1.3 |
| 1638 | RS08585 | protein | integrase | | | | | |
| H7A79_{—} | H7A79_{—} | hypothetical | PHAGE_Entero_c_1_NC_019706: | 7 | intact | ns | ns | 1.1 |
| 1639 | RS08590 | protein | prophage antirepressor | | | | | |
| H7A79_{—} | H7A79_{—} | helix-turn-helix | PHAGE_Mannhe_vB_MhM_{—} | 7 | intact | 1.5 | 1.1 | 1.4 |
| 1640 | RS08595 | transcriptional regulator | 1127AP1_NC_047750: holin | | | | | |
| H7A79_{—} | H7A79_{—} | DUF2303 family | PHAGE_Arthro_Bridgette_NC_048091: | 7 | intact | 1.9 | ns | 1.2 |
| 1643 | RS08610 | protein | tail tip protein | | | | | |
| H7A79_{—} | H7A79_{—} | hypothetical | n/a | 7 | intact | 1.7 | 1.2 | 1.6 |
| 1644 | RS08615 | protein | | | | | | |
| H7A79_{—} | H7A79_{—} | hypothetical | n/a | 7 | intact | 1.8 | 1.2 | 1.6 |
| 1645 | RS08620 | protein | | | | | | |
| H7A79_{—} | H7A79_{—} | hypothetical | n/a | 7 | intact | 1.4 | 1.0 | 1.5 |
| 1646 | RS08625 | protein | | | | | | |
| H7A79_{—} | H7A79_{—} | hypothetical | n/a | 7 | intact | ns | ns | 2.1 |
| 1649 | RS08635 | protein | | | | | | |
| H7A79_{—} | H7A79_{—} | hypothetical | PHAGE_Vibrio_vB_VpaM_MAR_NC_019722: | 7 | intact | 1.4 | 1.5 | 1.1 |
| 1652 | RS08655 | protein | putative exonuclease | | | | | |
| H7A79_{—} | H7A79_{—} | HicA family | PHAGE_Mannhe_vB_MhS_{—} | 7 | intact | 2.0 | ns | 2.0 |
| 1654 | RS08665 | toxin | 535AP2_NC_028853: | | | | | |
| | | | toxin HicA | | | | | |

TABLE 4

| Refseq | genbank | | | | | | | | Bastion | |
|---|---|---|---|---|---|---|---|---|---|---|
| locus | locus | | FC | FC | FC | | signalP | Outcyte | Secretion | |
| tag | tag | wp_ID | wk8 | wk6 | oral | annotation | result | prediction | prediction | % ID |
| H7A79_{—} | H7A79_{—} | NZ_{—} | −2.0052758 | −3.6884342 | −3.046717 | predicted | lipo- | signal | yes | nwe(62.6) |
| RS01025 | 0186 | CP060414.1_{—} | | | | secreted | protein | peptide | type iii | nei(62.6), |
| | | prot_{—} | | | | protein | | | | ncs(59.6), |
| | | WP_135035093.1_{—} | | | | | | | | ngo(58.3), |
| | | 194 | | | | | | | | nel(50.9), |
| | | | | | | | | | | nmj(52.8), |
| | | | | | | | | | | nsc(52.8) |
| H7A79_{—} | H7A79_{—} | NZ_{—} | −6.9897874 | −7.7951023 | −8.2924231 | putative | lipo- | signal | yes | nwe(54.9), |
| RS01030 | 0187 | CP060414.1_{—} | | | | lipo- | protein | peptide | type iv | ngk(47.6), |
| | | prot_{—} | | | | protein | | | | ngo(47.6), |
| | | WP_135035108.1_{—} | | | | | | | | ncz(47.4), |
| | | 195 | | | | | | | | nmj(50.7), |
| | | | | | | | | | | nsi(50.7) |
| H7A79_{—} | H7A79_{—} | Z_{—} | −2.39352072 | −2.49212027 | −2.74807151 | putative | other | trans- | no | nel(62.2), |
| RS02215 | 0417 | CP060414.1_{—} | | | | membrane | | membrane | | nfv(55.0), |
| | | prot_WP_{—} | | | | protein | | | | nfs(55.0), |
| | | 135034309.1_{—} | | | | | | | | ncz(52.6), |
| | | 432 | | | | | | | | nma(52.6), |
| H7A79_{—} | H7A79_{—} | NZ_{—} | −2.85319108 | −1.50748063 | −2.43799395 | putative | other | trans- | no | nwe(51.8), |
| RS02885 | 0540 | CP060414.1_{—} | | | | membrane | | membrane | | nbl(46.7), |
| | | prot_{—} | | | | protein | | | | nsc(46.6), |
| | | WP_{—} | | | | | | | | nfv(43.3), |
| | | 187001007.1_{—} | | | | | | | | nsf(43.3), |
| | | 567 | | | | | | | | nla(41.5), |
| | | | | | | | | | | ncz(40.4), |
| | | | | | | | | | | nma(40.4), |
| | | | | | | | | | | ngo(38.9) |
| H7A79_{—} | H7A79_{—} | NZ_{—} | −4.77702015 | −3.4402739 | −4.59693816 | conserved | other | trans- | yes, | nwe(98.8), |
| RS05395 | 1027 | CP060414.1_{—} | | | | hypothetical | | membrane | type iv | nmw(93.7) |
| | | prot_{—} | | | | integral | | | | |
| | | WP_{—} | | | | membrane | | | | |
| | | 187001312.1_{—} | | | | protein, | | | | |
| | | 1068 | | | | abc-2 | | | | |
| | | | | | | transporter | | | | |
| | | | | | | protein | | | | |
| H7A79_{—} | H7A79_{—} | NZ_{—} | −1.15475302 | −1.29101514 | −1.03301511 | putative | other | trans- | no | nwe(84.2), |
| RS06305 | 1206 | CP060414.1_{—} | | | | membrane | | membrane | | nel(76.3), |
| | | prot_{—} | | | | protein | | | | nla(74.7), |
| | | WP_{—} | | | | | | | | nmc(73.7), |
| | | 135037421.1_{—} | | | | | | | | nme(73.7), |
| | | 1246 | | | | | | | | nsi(72.6), |
| | | | | | | | | | | ncz(73.7), |
| | | | | | | | | | | ngo(73.7), |
| | | | | | | | | | | nfv(72.6), |
| | | | | | | | | | | nsf(72.6) |
| H7A79_{—} | H7A79_{—} | NZ_{—} | −1.88623188 | −2.49661859 | −3.08738972 | secreted | other | trans- | no | nwe(61.0), |
| RS08300 | 1584 | CP060414.1_{—} | | | | protein | | membrane | | nbl(62.1), |
| | | prot_{—} | | | | | | | | nma(51.4), |
| | | WP_{—} | | | | | | | | nme(51.4), |
| | | 187001671.1_{—} | | | | | | | | nla(52.1), |
| | | 1633 | | | | | | | | ncz(51.4), |
| | | | | | | | | | | ngo(51.4), |
| | | | | | | | | | | nsc(51.4), |
| | | | | | | | | | | nmj(52.1) |
| H7A79_{—} | H7A79_{—} | NZ_{—} | −3.08536118 | −1.05723068 | −1.21067391 | putative | sec/spi | signal | III | nbl(41.8), |
| RS12790 | 2449 | CP060414.1_{—} | | | | secreted | | peptide | | ncz(32.6), |
| | | prot_{—} | | | | protein | | | | nmc(29.4), |
| | | WP_{—} | | | | | | | | nme(29.4), |
| | | 187000495.1_{—} | | | | | | | | nla(29.4), |
| | | 2498 | | | | | | | | ngo(29.7), |
| | | | | | | | | | | nsc(34.8), |
| | | | | | | | | | | nsi(34.8) |
| H7A79_{—} | H7A79_{—} | NZ_{—} | −6.49558094 | −3.44596847 | −2.92640242 | putative | other | trans- | no | nwd(66.2), |
| RS13830 | 2647 | CP060414.1_{—} | | | | membrane | | membrane | | nbc(63.8), |
| | | prot_{—} | | | | protein | | | | nme(64.4), |
| | | WP_{—} | | | | | | | | nla(63.8), |
| | | 187000600.1_{—} | | | | | | | | ngo(63.8), |
| | | 2710 | | | | | | | | ncz(61.7), |
| | | | | | | | | | | nsf(55.7), |
| | | | | | | | | | | nfv(55.7) |
| H7A79_{—} | H7A79_{—} | NZ_{—} | −4.30699365 | −5.35823746 | −6.44729311 | | lipo- | signal | I | nel(52.0), |
| RS07235 | 1377 | CP060414.1_{—} | | | | | protein | peptide | | nbc(50.6), |
| | | prot_{—} | | | | | | | | ngo(51.2), |
| | | WP_{—} | | | | | | | | nma(48.9), |
| | | 187001543.1_{—} | | | | | | | | nme(48.9) |
| | | 1425 | | | | | | | | |
| H7A79_{—} | H7A79_{—} | NZ_{—} | −5.0624837 | −4.0374687 | −4.0764794 | | sec/spi | signal | IV | nbl(51.4), |
| RS08000 | 1524 | CP060414.1_{—} | | | | | | peptide | | nzl(63.8), |
| | | prot_{—} | | | | | | | | nsc(51.4), |
| | | WP_{—} | | | | | | | | nsi(51.4), |
| | | 135036472.1_{—} | | | | | | | | nsf(49.0), |
| | | 1572 | | | | | | | | nfv(47.6), |
| | | | | | | | | | | nel(39.5), |
| | | | | | | | | | | ncz(36.4), |
| | | | | | | | | | | nla(33.6), |
| | | | | | | | | | | nme(33.0), |
| | | | | | | | | | | ngo(32.0) |
| H7A79_{—} | H7A79_{—} | \|NZ_{—} | −4.9635896 | −4.6102999 | −5.7554366 | | other | transmembrane | | nwe(86.8), |
| RS01505 | 0280 | CP060414.1_{—} | | | | | | | | ncz(85.3), |
| | | prot_{—} | | | | | | | | ngo(85.3), |
| | | WP_{—} | | | | | | | | nla(83.8), |
| | | 187000847.1_{—} | | | | | | | | nmd(83.8), |
| | | 289 | | | | | | | | nbl(83.8), |
| | | | | | | | | | | nmj(78.8), |
| | | | | | | | | | | nsc(78.8), |
| | | | | | | | | | | nfv(76.5), |
| | | | | | | | | | | nsf(76.5), |
| | | | | | | | | | | nel(69.2) |
| H7A79_{—} | H7A79_{—} | NZ_{—} | −2.0561022 | −3.1242962 | −2.6209535 | | other | transmembrane | | nzl(75.7), |
| RS14150 | 2705 | CP060414.1_{—} | | | | | | | | nfv(75.2), |
| | | prot_{—} | | | | | | | | nsf(75.7), |
| | | WP_{—} | | | | | | | | nme(75.7), |
| | | 135034727.1_{—} | | | | | | | | nbl(73.4), |
| | | 2774 | | | | | | | | ncz(76.1), |
| | | | | | | | | | | nla(75.2), |
| | | | | | | | | | | ngo(73.4), |
| | | | | | | | | | | nmc(75.2), |
| | | | | | | | | | | nel(73.1), |
| | | | | | | | | | | nsc(70.9), |
| | | | | | | | | | | nmj(74.5) |

Hypothetical proteins were further analyzed to determine membrane localization and/or putative secretion using SignalP 5.0, Outcyte and BastionX. SignalP 5.0 (SignalP 5.0 improves signal peptide predictions using deep neural networks. José Juan Almagro Armenteros, Konstantinos D. Tsirigos, Casper Kaae Sønderby, Thomas Nordahl Petersen, Ole Winther, Søren Brunak, Gunnar von Heijne and Henrik Nielsen. Nature Biotechnology, 37, 420-423, doi: 10.1038/s41587-019-0036-z (2019); Outcyte: Zhao, L., Poschmann, G., Waldera-Lupa, D. et al. OutCyte: a novel tool for predicting unconventional protein secretion. Sci Rep 9, 19448 (2019). https://doi.org/10.1038/s41598-019-55351-z BastionHub (Wang J et al. BastionHub: a universal platform for integrating and analyzing substrates secreted by Gram-negative bacteria. Nucleic Acids Research 2020)

TABLE 5
>lcl|NZ_CP060414.1_cds_WP_135035093.1_194 [locus_tag = H7A79_RS01025]
[protein = hypothetical protein] [protein_id= WP_135035093.1]
[location = complement(219980..220303)] [gbkey = CDS] (SEQ ID NO: 1)
ATGAATAAATTAATATCCGCCTTATTTGCAGCTTGTGTTTTGGCTGCCTGCGCATC
GCCGAACTCTGCCGGCAATATGGCGATCGGCGATGATTCTGCCGCCATTAAGGCG
GGAAGAAACCGAATGTCTGCCGAGTTGAGCCGGTCTGAATTGGAGCAGCAGCGC
CGCCAAAGAACCAACACGGCAGAGGAAATCGCTTTAGAGGAGCAAAAGCGCAG
AAACAAACACGAAGGCATCAGGGATACCATGCGCACCACTTCGGGCGGCCTGGG
TGTGATTAATGAGGCAATCGGCACCATACGCAACCTACGCTGGGGCTTTTAA
>lcl|NZ_CP060414.1_cds_WP_135035108.1_195 [locus_tag = H7A79_RS01030]
[protein = hypothetical protein] [protein_id = WP_135035108.1]
[location = complement(220360..220614)] [gbkey = CDS] (SEQ ID NO: 2)
ATTTTTGTGAACAATTTGTTAATCCCCGCTTTCGCTGCCGTGCTGCTGCTTGGCGG
CTGCACTTATGCCGACCAACGCTTTGTTTCCTCCCAATCCCAGCAACAGCAGCTT
TATGAAAACAGGCTCGCTATCCAGAAAGCAGAGCGGGAGGAGCGGGCTGAGCG
CAGAGCCGAACGCCGCGAAGAGATGATGGATGAGGCCGATGCCGTCAACCGCGC
ATATAAAGGGCTTAACGGAGTTTACCTTATCCGCTGA
>lcl|NZ_CP060414.1_cds_WP_135034309.1_432 [locus_tag = H7A79_RS02215]
[protein = hypothetical protein] [protein_id = WP_135034309.1]
[location = complement(464334..464744)] [gbkey = CDS] (SEQ ID NO: 3)
ATGTTTAAACGCCCTGAAGAGTTGATTGTGGCTGTGCTGGCCGCCTTGTGGGTGG
TGTTGGCCTATCTGGCCGCCGATTATCTCGGTGCCCCCGCGCAAACCAAGTTGCT
GATTTCTGCGCTGACGCTGATTTGTACCGCCCTGTGCTTCATACTGTGGCAGCGC
AACCTGAGCCGGGCGGTTTGGCCGTTTTTTCTGGGAATGCTGACCGCCTGTTGGT
GGCCTTATCTCGATTGGATGGCGGTGCGCGATATTCTGGTGCCGGGCGCAGAGTC
TTCGGCCATTATCATCAACAAGCCCTGGTATGCGGGCTGGACTTTTAAAACCATT
CTTGCCGCCGCGCCCGTGGTGCTGGGCTATGCGGTAAAGTGGAAACTGCACAGC
AAACACAAGCATAAAGCGGTGGATTAG
>lcl|NZ_CP060414.1_cds_WP_187001007.1_567 [locus_tag = H7A79_RS02885]
[protein = hypothetical protein] [protein_id = WP_187001007.1] [location = 588020..588622]
[gbkey = CDS] (SEQ ID NO: 4)
ATGGCCGGTTGGGATGTATGGATTTCGGTTGTTTATATTGCCGTGTTTTTCACCGC
TGCTTTTAAAGCGGGAGAGTTTCAATGGCTGTGGGGAAGCGTGCTGCTCTGGTTT
GGGTTCGGCCTTGCCGGCGCACGCCTTCTGCCTGGAATATGGGGTATCATCCACT
TGGCTCCGCTTTATCCTCCCCATCTCTATATCATACCGGCCAGCCTGTTTTTCTTTG
CAAACCATTGGAAAAAAGTGCCTGAAAAGAAGATGTGGCAGGCCGAAAATGCC
AACGGTTTTATCAGCTTGTTTGCAGTAAGCGGTATGCTGATGGGCATCGTGTCTG
CTGTGTTGGCGTTAGTGGTGCTCAGCCGCTTTCCGGCGGGCATAACCCCTTATGTT
TTGCCTGTGCTGCTGCAGCTTTATGCTTTCGAACCGGCGTATTGGTTCGGAATGCA
GGCTGTGATCATGGTGGTTTTTTATCTGCACCGCAAAGTTATTGCGGGCGAGGCG
GCAAACCGTTTCAGCCGCCTCCAGCTGCAACTGGGTTTTCTGTTGGCCTTATTGTT
TCAGACGGCCTGCACAGTTGTATTGCTGTCAGATATACGTTACGGTTGA
>lcl|NZ_CP060414.1_cds_WP_187001312.1_1068 [locus_tag = H7A79_RS05395]
[protein = hypothetical protein] [protein_id = WP_187001312.1] [location = 1071533..1072021]
[gbkey = CDS] (SEQ ID NO: 5)
ATGAAAAAATACCTGCCCTTTTTACGTTTTGTGCTGATTGCCTGTATATCGGCATT
CTTGGCGTGGAAAGGGCAGCCTTTTGTTCATGGAAACGAGAAAGCAGTTGATTTA
ATTATCAACGTATTTGCAATCTTGGCCGGTTTTCTGATTGCGATTATGACGCTGTT
TAGTGATATGCGGTTTGATGAAGATGCGAATTGGCGGCAAATTCAAATTCGTGAA
GGCGTACAAGAACAAAGATATATCAAACATTCCATGCTCTTTTACACTTATCTGG
CAGTATTGGTTTGTGTTTTTCTTGTTATATTGTTGGCTCATAAAGAAGAATATAAA
AATGGACAAATAATTTTTTGGCTAGAGCGGGGATATTTATTCTTAGCCTGCATGT
CCATTTTTTACTCTGTATTTTTACCCGGAAACCTAATTAAAAGCCGGAAAGAAGA
GTTTCAAAAACTCATGGATAAAAAGAAACCCAAATATCCGAATTGA
>lcl|NZ_CP060414.1_cds_WP_135037421.1_1246 [locus_tag = H7A79_RS06305]
[protein = hypothetical protein] [protein_id = WP_135037421.1]
[location = complement(1238460..1238747)] [gbkey = CDS] (SEQ ID NO: 6)
ATGAATAAAGAATTGCTCGGCATTGTTTTTCTGCCCGCCGGCGTCATCAGCATGT
GCATGGCGGCATTGTGGCAGATGTATGTGGTGATCACCGAAAGCTACATGCTTAA
CCGCTTCAAAGACAAGCAATTGGTGTGGGTAGTGGCCGTGCTGTTTTTCAGCTTC
AGCACTGCCGTTTACTGGTTTTGCCCGAATGCGCGCAAAAAAGGCATATTGTTTC
TGCTGCTGGGTGCGGGCGGCGCGGTGATGTATGGCTTGGCCAGAATATGGCTGCC
GTTCGGCAAATAA
>lcl|NZ_CP060414.1_cds_WP_187001671.1_1633 [locus_tag = H7A79_RS08300]
[protein = hypothetical protein] [protein_id = WP_187001671.1] [location = 1647157..1647594]
[gbkey = CDS] (SEQ ID NO: 7)
TTGAAACTCAATCGAAAACAAGCCGTTTGGGCGGGCATATTGCTGTTGGCGTTTG
CCGCCGTAAAAATAGCGGCACTGCTGTGGTGGCAGAGCCGCCAGCCCGCCGTTA
CCTCCGTTGCCGAATCTGCCTGCAACGTACACGCGGGCTGCACGCTGCCCAACGG
AGCGCAAGTGAAATTCAGCAGCAAAGTGGCCGCCAAAGCGCCGTTCGACATCAC
CCTGCACAACGTGCCGGCGCAAACGGCCGAAGTGTTTGTGAGTTTTTCGATGCGC
GGTATGGACATGGGCTTCAACCGCTACAAACTGCTGCGCCGGCCTGACGGCTCGT
GGGCGGCACCGCAAATCCGTCTGCCCGTGTGCGTGCAGAACCGCCGTGATTATCT
GGCCGACATCCATATCGGCGGCGAGGTGTTCCAAGTGGGCTTTACCGCCGAGTG
A
>lcl|NZ_CP060414.1_cds_WP_187000495.1_2498 [locus_tag-H7A79_RS12790]
[protein = hypothetical protein] [protein_id = WP_187000495.1] [location = 2559932..2560207]
[gbkey = CDS] (SEQ ID NO: 8)
ATGCAAAGCACGCAACGCAAAGGTTTGGTGTTGGTCAGCACATTGGCCGCAATG
GCTGCAGCATTCTCTTCCGGCCACACGGAGGCCCAATCCGCCGAAATGCCGCTGT
CGGAGTTGGGCTGCGATAAATTGAATGTATTGCGAATCACGCAAATGAACGGTA
AACAACAAGTTTTTGCACGCGAATGCGCCGCTGCAGAAGCCGCCCAAGCATGGG
AACAGTCTTACGGCGCACTTGACTCCGAAAGTTTGATTGCGAGTGTGATATACGA
ATAG
>lcl|NZ_CP060414.1_cds_WP_187000600.1_2710 [locus_tag = H7A79_RS13830]
[protein = hypothetical protein] [protein_id = WP_187000600.1] [location = 2781169..2781615]
[gbkey = CDS] (SEQ ID NO: 9)
ATGAGCGTTTATTCTGTTGCACATATCATACATTTATTTTGCGCCATCGTTTTTGT
GGGCGGCGTTTTCTTTGAAACGCTGGTGTTGTCGGCTATACACAGCAAAGATGTA
CCGAGGGAGGCGCGCAGAACGGTAGAAAAAGCCATTATGCGGCGGGCGGTGCG
GGTGATGCCGTGGGTGGTGGGCAGTGTGTTTCTGTCGGGGTTGACAATGGCACAC
CGTTATGCTGCCGCATTCGCCGCACCATTTTCCGCCCCGTTCAACACTCAGCTGTG
TTTGAAAGCGTTGCTGGCGTTTGGTGTGTTAGCGCACTTTCTGATTGCCATAATCA
AAATGCGGCGCAATACACTCACAGCAGCTTGGTCAAAATATATCCATCTGGCGGT
ATTTTGCCAGATGGTGTTGATTGTGCTTCTGGCGAAAACCATGTTTTATATAGCTT
GGTAG
>lcl|NZ_CP060414.1_cds_WP_187001543.1_1425 [locus_tag = H7A79_RS07235]
[protein = hypothetical protein] [protein_id = WP_187001543.1]
[location = complement(1412942..1413202)] [gbkey = CDS] (SEQ ID NO: 10)
ATGAAAACCACAACTTCTACTGCTTTGGCCGCATTGGCCGGTGCGCTGGCACTGG
CGGGTTGTCAGGCCGGTTCTGCCCCCTCTGCTCCGATTGCAGCCTCTGGGCAGCA
TAAAGTGGGCGAGGGAAAATGCGGCGCAAGCGGCAAAGCTGCCGAAGGTAAAT
GCGGCGCAACATCGCACAATGGCAAGGCTGCCGAAGGCAAATGCGGCGCAGCAT
CGCACAGCGGTAAGTCTGCCGAAGGTAAATGCGGCAGTAAATGA
>lcl|NZ_CP060414.1_cds_WP_135036472.1_1572 [locus_tag = H7A79_RS08000]
[protein = DUF4124 domain-containing protein] [protein_id = WP_135036472.1]
[location = 1576014..1576457] [gbkey = CDS] (SEQ ID NO: 11)
ATGCACATTACCCGCACCGTCTATACTGCCGCTCTTCTCTGCCTGCTGCCTGCGCA
ATTTGCCGCTGCCCAGATTTACACCTGGAAAGGCAAAAACGGCAGCACCAGTTA
TTCAGATGTGCCGCACAACCTGAAAATTTCCAACGCATCAACCATCAACGTGCGC
ACCCGCACCGTTACCCCGCCGCCCGCCGCCCAAACCGCAGAAAGCGCAGGCGAA
GGCTCGCTGGCCGACCAACAAAAACAGCTCAACGATGCCGTCAGCCGTGAAAAT
AAAAAAATCGAAGAGCAAAACAAAAAAATCGAAGAACAAAACCGCCTGCAAAA
AGAAGACAACTGCAAAACCGCGCGCCTCAACCGCCAGTTTGCCGAAACCGCCCG
CGTGGCCAACCGCGATGAATTTATCAAGCGTTACGATGCCGATATCGGCAAGTTC
TGCAATTAG
>lcl|NZ_CP060414.1_cds_WP_187000847.1_289 [locus_tag = H7A79_RS01505]
[protein = DUF2788 domain-containing protein] [protein_id = WP_187000847.1]
[location = 328706..328912] [gbkey = CDS] (SEQ ID NO: 12)
ATGGATGAAGCGGTGTTTGCTGATTGGGCGTTGAAAATCTGCCTGGGCGGGCTGG
TGGTTTTTTTGGGATTTATCGTTTGGAATCTGGGTAAGGAATCGAAAGCGGGAAA
ATTCGGTATGTTTGTGCTGTTTTTGGTGTTGGGGCTGGGTGTGTTCGGCTTTCTTTT
CAAAAATGTGTTAATCGAATTTTTGGTGTTGTCGAAATAA
>lcl|NZ_CP060414.1_cds_WP_135034727.1_2774 [locus_tag = H7A79_RS14150]
[protein = DUF502 domain-containing protein] [protein_id = WP_135034727.1]
[location = 2834287..2834985] [gbkey = CDS] (SEQ ID NO: 13)
ATGGCAAAAAATGCGGCTGAAAGCGGCGGCATCTCCAGCGCATTGAAAAAATAC
CTGATTACAGGTGTTTTGGTGTGGCTGCCGATAGCCGTAACCGTGTGGGTGATCA
CCTATATCGTTTCCGCATCCGACCGGCTGTTCCAGCTGCTGCCGCTCAAATGGCA
GCCGGCCAACCTGATAGGCTTCAACATTCCCGGCTTGGGCGTGATTGTGGCGGTG
GCCGTGTTGTTTGCCACCGGCCTGTTTGCCGCCAATATGCTGGGCAGGCAGATTA
TTGCGGTATGGGACAACATGCTCGGCCGCATTCCTGTGGTGAAATCGATTTATTC
CAGCGTAAAAAAAGTTTCGGAATCACTGTTTTCCGACAACAGCCGCTCATTCAAA
ACCCCTGTTTTAGTGCCGTTTCCCCAGCCGGGCATCTGGACGATCGCTTTTGTGTC
GGGCAGCCTTGCCGATGCAGTGGGTCGCAGCCTCGATAATGGCGAAGAATACCT
TTCCGTTTATGTGCCGACCACGCCCAACCCCACCGGCGGTTATTATGTGATGGTA
AAAAAAAGCGATGTGCGCGAGCTGGATATGAGCGTGGACGATGCCTTGAAATAT
GTGATTTCGCTAGGTATGGTAATGCCGGAAGACCGGTCGGCGAAAGCCCTGCCC
GATGCGCAGAAGCCGTCTGAACACCCCGCAGGGCAAGAATAA

TABLE 6
>lcl|NZ_CP060414.1_prot_WP_135035093.1_194 [locus_tag-H7A79_RS01025]
[protein-hypothetical protein] [protein_id = WP_135035093.1]
[location = complement(219980 . . . 220303)] [gbkey = CDS] (SEQ ID NO: 14)
MNKLISALFAACVLAACASPNSAGNMAIGDDSAAIKAGRNRMSAELSRSELEQQRR
QRTNTAEEIALEEQKRRNKHEGIRDTMRTTSGGLGVINEAIGTIRNLRWGF
>lcl|NZ_CP060414.1_prot_WP_135035108.1_195 [locus_tag-H7A79_RS01030]
[protein-hypothetical protein] [protein_id = WP_135035108.1]
[location = complement(220360 . . . 220614)] [gbkey = CDS] (SEQ ID NO: 15)
MFVNNLLIPAFAAVLLLGGCTYADQRFVSSQSQQQQLYENRLAIQKAEREERAERRA
ERREEMMDEADAVNRAYKGLNGVYLIR
>lcl|NZ_CP060414.1_prot_WP_135034309.1_432 [locus_tag-H7A79_RS02215]
[protein = hypothetical protein] [protein_id = WP_135034309.1]
[location = complement(464334 . . . 464744)] [gbkey = CDS] (SEQ ID NO: 16)
MFKRPEELIVAVLAALWVVLAYLAADYLGAPAQTKLLISALTLICTALCFILWQRNL
SRAVWPFFLGMLTACWWPYLDWMAVRDILVPGAESSAIIINKPWYAGWTFKTILAA
APVVLGYAVKWKLHSKHKHKAVD
>lcl|NZ_CP060414.1_prot_WP_187001007.1_567 [locus_tag = H7A79_RS02885]
[protein-hypothetical protein] [protein_id = WP_187001007.1] [location = 588020 . . . 588622]
[gbkey = CDS] (SEQ ID NO: 17)
MAGWDVWISVVYIAVFFTAAFKAGEFQWLWGSVLLWFGFGLAGARLLPGIWGIIHL
APLYPPHLYIIPASLFFFANHWKKVPEKKMWQAENANGFISLFAVSGMLMGIVSAVL
ALVVLSRFPAGITPYVLPVLLQLYAFEPAYWFGMQAVIMVVFYLHRKVIAGEAANR
FSRLQLQLGFLLALLFQTACTVVLLSDIRYG
>lcl|NZ_CP060414.1_prot_WP_187001312.1_1068 [locus_tag = H7A79_RS05395]
[protein-hypothetical protein] [protein_id = WP_187001312.1] [location = 1071533 . . . 1072021]
[gbkey = CDS] (SEQ ID NO: 18)
MKKYLPFLRFVLIACISAFLAWKGQPFVHGNEKAVDLIINVFAILAGFLIAIMTLFSDM
RFDEDANWRQIQIREGVQEQRYIKHSMLFYTYLAVLVCVFLVILLAHKEEYKNGQIIF
WLERGYLFLACMSIFYSVFLPGNLIKSRKEEFQKLMDKKKPKYPN
>lcl|NZ_CP060414.1_prot_WP_135037421.1_1246 [locus_tag-H7A79_RS06305]
[protein = hypothetical protein] [protein_id = WP_135037421.1]
[location = complement(1238460 . . . 1238747)] [gbkey = CDS] (SEQ ID NO: 19)
MNKELLGIVFLPAGVISMCMAALWQMYVVITESYMLNRFKDKQLVWVVAVLFFSF
STAVYWFCPNARKKGILFLLLGAGGAVMYGLARIWLPFGK
>lcl|NZ_CP060414.1_prot_WP_187001671.1_1633 [locus_tag-H7A79_RS08300]
[protein = hypothetical protein] [protein_id = WP_187001671.1] [location = 1647157 . . . 1647594]
[gbkey = CDS] (SEQ ID NO: 20)
MKLNRKQAVWAGILLLAFAAVKIAALLWWQSRQPAVTSVAESACNVHAGCTLPNG
AQVKFSSKVAAKAPFDITLHNVPAQTAEVFVSFSMRGMDMGFNRYKLLRRPDGSW
AAPQIRLPVCVQNRRDYLADIHIGGEVFQVGFTAE
>lcl|NZ_CP060414.1_prot_WP_187000495.1_2498 [locus_tag = H7A79_RS12790]
[protein = hypothetical protein] [protein_id = WP_187000495.1] [location = 2559932 . . . 2560207]
[gbkey = CDS] (SEQ ID NO: 21)
MQSTQRKGLVLVSTLAAMAAAFSSGHTEAQSAEMPLSELGCDKLNVLRITQMNGK
QQVFARECAAAEAAQAWEQSYGALDSESLIASVIYE
>lcl|NZ_CP060414.1_prot_WP_187000600.1_2710 [locus_tag-H7A79_RS13830]
[protein-hypothetical protein] [protein_id = WP_187000600.1] [location = 2781169 . . . 2781615]
[gbkey-CDS] (SEQ ID NO: 22)
MSVYSVAHIIHLFCAIVFVGGVFFETLVLSAIHSKDVPREARRTVEKAIMRRAVRVMP
WVVGSVFLSGLTMAHRYAAAFAAPFSAPFNTQLCLKALLAFGVLAHFLIAIIKMRRN
TLTAAWSKYIHLAVFCQMVLIVLLAKTMFYIAW
>lcl|NZ_CP060414.1_prot_WP_187001543.1_1425 [locus_tag-H7A79_RS07235]
[protein = hypothetical protein] [protein_id = WP_187001543.1]
[location = complement(1412942 . . . 1413202)] [gbkey = CDS] (SEQ ID NO: 23)
MKTTTSTALAALAGALALAGCQAGSAPSAPIAASGQHKVGEGKCGASGKAAEGKC
GATSHNGKAAEGKCGAASHSGKSAEGKCGSK
>lcl|NZ_CP060414.1_prot_WP_135036472.1_1572 [locus_tag = H7A79_RS08000]
[protein = DUF4124 domain-containing protein] [protein_id = WP_135036472.1]
[location = 1576014 . . . 1576457] [gbkey = CDS] (SEQ ID NO: 24)
MHITRTVYTAALLCLLPAQFAAAQIYTWKGKNGSTSYSDVPHNLKISNASTINVRTR
TVTPPPAAQTAESAGEGSLADQQKQLNDAVSRENKKIEEQNKKIEEQNRLQKEDNC
KTARLNRQFAETARVANRDEFIKRYDADIGKFCN
>lcl|NZ_CP060414.1_prot_WP_187000847.1_289 [locus_tag = H7A79_RS01505]
[protein = DUF2788 domain-containing protein] [protein_id = WP_187000847.1]
[location = 328706 . . . 328912] [gbkey = CDS] (SEQ ID NO: 25)
MDEAVFADWALKICLGGLVVFLGFIVWNLGKESKAGKFGMFVLFLVLGLGVFGFLF
KNVLIEFLVLSK
>lcl|NZ_CP060414.1_prot_WP_135034727.1_2774 [locus_tag = H7A79_RS14150]
[protein = DUF502 domain-containing protein] [protein_id = WP_135034727.1]
[location = 2834287 . . . 2834985] [gbkey = CDS] (SEQ ID NO: 26)
MAKNAAESGGISSALKKYLITGVLVWLPIAVTVWVITYIVSASDRLFQLLPLKWQPA
NLIGFNIPGLGVIVAVAVLFATGLFAANMLGRQIIAVWDNMLGRIPVVKSIYSSVKKV
SESLFSDNSRSFKTPVLVPFPQPGIWTIAFVSGSLADAVGRSLDNGEEYLSVYVPTTPN
PTGGYYVMVKKSDVRELDMSVDDALKYVISLGMVMPEDRSAKALPDAQKPSEHPAGQE

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. The following references are referenced within this application and are herein incorporated by reference in all entireties:

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A vaccine composition, comprising:
a) at least a portion of a plurality of nucleic acids selected from the group consisting of SEQ ID NOs: 1-13 or at least a portion of a plurality of polypeptides selected from the group consisting of SEQ ID NOs: 14-26; and
b) a pharmaceutically acceptable carrier.

2. The composition of claim 1, wherein said portion is at least 10 nucleotides or 5 amino acids.

3. The composition of claim 1 or 2, wherein said portion is at least 50 nucleotides or 10 amino acids.

4. The composition of any of the preceding claims, wherein said at least a portion is the entire sequence.

5. The composition of any of the preceding claims, wherein said nucleic acid is mRNA, DNA, or cDNA.

6. The composition of any of the preceding claims, wherein said nucleic acid is in a vector.

7. The composition of claim 6, wherein said vector is an adenoviral vector.

8. The composition of any of the preceding claims, wherein said pharmaceutically acceptable carrier is an adjuvant.

9. The composition of any of the preceding claims, wherein said pharmaceutically acceptable carrier is a lipid.

10. The composition of any of the preceding claims, wherein said plurality of nucleic acid or polypeptide sequences is two or more.

11. A method of generating an immune response, comprising:
administering the vaccine composition of any one of claims 1 to 10 to a subject in need thereof.

12. The method of claim 11, wherein said immune response generates immunity against infection by a Neisseria sp.

13. The method of claim 12, wherein said Neisseria sp. is Neisseria gonorrhoeae (Ngo).

14. The method of claim 12, wherein said Neisseria sp. is Neisseria meningitidis (Nine).

15. The use of the vaccine composition of any one of claims 1 to 10 to generate an immune response is a subject.

16. The use of the vaccine composition of any one of claims 1 to 10 to prevent infection against a Neisseria sp. in a subject.

17. The vaccine composition of any one of claims 1 to 10 for use in generating an immune response is a subject.

18. The vaccine composition of any one of claims 1 to 10 for use in preventing infection against a Neisseria sp. in a subject.

19. A composition comprising an agent that inhibits the expression or one or more activities of a polypeptide selected from the group consisting of SEQ ID NOs: 14-26.

20. The composition of claim 19, wherein said agent is selected from the group consisting of a nucleic acid, a protein, an antibody, and a small molecule.

21. The composition of claim 20, wherein said nucleic acid is selected from the group consisting of an siRNA, an antisense nucleic acid, and miRNA, a guide RNA, and a shRNA.

22. A method of treating or preventing infection by a Neisseria sp., comprising administering the composition of any one of claims 19 to 21 to a subject in need thereof.

23. The use of the composition of any one of claims 19 to 21 to treat or prevent infection by Neisseria sp. in a subject.

24. The composition of any one of claims 19 to 21 to treat or prevent infection by Neisseria sp. in a subject.

25. A method of screening a compound, comprising:
a) contacting a polypeptide selected from the group consisting of SEQ ID NOs: 14-26 with a test compound; and
b) assaying the effect of said test compound on the expression or one or more activities of said polypeptide.

26. A composition, comprising:
at least a portion of a nucleic acid selected from the group consisting of SEQ ID NOs: 1-13 or at least a portion of a polypeptide selected from the group consisting of SEQ ID NOs: 14-26.
